(19) United States
(12) Patent Application Publication
(10) Pub. No.: US 2023/0267699 A1
(43) Pub. Date: Aug. 24, 2023

(54) METHODS AND APPARATUSES FOR TELE-MEDICINE

(71) Applicant: BFLY OPERATIONS, INC., Burlington, MA (US)

(72) Inventors: Maxim Zaslavsky, San Diego, CA (US); Matthew de Jonge, Brooklyn, NY (US); David Elgena, Orlando, FL (US); Patrick Temple, Brooklyn, NY (US); Jason Quense, Somerville, NJ (US); Aditya Ayyakad, Freehold, NJ (US)

(73) Assignees: BFLY OPERATIONS, INC., Burlington, MA (US); BFLY OPERATIONS, INC., Burlington, MA (US)

(21) Appl. No.: 18/137,049

(22) Filed: Apr. 20, 2023

Related U.S. Application Data
(62) Division of application No. 16/735,019, filed on Jan. 6, 2020.
(60) Provisional application No. 62/933,306, filed on Nov. 8, 2019.
(60) Provisional application No. 62/789,394, filed on Jan. 7, 2019.

(51) Int. Cl. G06V 10/10; A61B 8/00; G06T 19/00; G16H 40/67; G06T 19/20

(52) U.S. Cl. CPC G06V 10/17; A61B 8/565; G06T 19/006; G16H 40/67; G06T 19/20; G06T 19/003; G06T 2219/2016

(57) ABSTRACT

Aspects of the technology described herein relate to operator processing devices and instructor processing device for tele-medicine. The instructor processing device may be configured to receive, from an instruction interface, a selection of an instruction for moving an ultrasound device. The operator processing device may be configured to determine a pose of the ultrasound device relative to the operator processing device. The instructor processing device and the operator processing device may be configured to display in an operator video, based on the pose of the ultrasound device relative to the operator processing device and based on the selected instruction, a directional indicator for moving the ultrasound device. The instructor processing device may also be configured to display, based on the pose of the ultrasound device relative to the operator processing device, orientation indicators in the instruction interface and/or the operator video.

### CROSS-REFERENCE TO RELATED APPLICATIONS

This application is a divisional of and claims the benefit of priority under 35 U.S.C. § 120 to U.S. patent application Ser. No. 16/735,019, filed on Jan. 6, 2020 and entitled “METHODS AND APPARATUSES FOR TELE-MEDICINE”, which is hereby incorporated herein by reference in its entirety.

U.S. patent application Ser. No. 16/735,019 claims the benefit under 35 U.S.C. § 119(e) of U.S. Provisional Application Ser. No. 62/933,306, filed on Nov. 8, 2019 and entitled “METHODS AND APPARATUSES FOR TELE-MEDICINE”, which is hereby incorporated herein by reference in its entirety.

U.S. patent application Ser. No. 16/735,019 also claims the benefit under 35 U.S.C. § 119(e) of U.S. Provisional Application Ser. No. 62/789,394, filed on Jan. 7, 2019 and entitled “METHODS AND APPARATUSES FOR TELE-MEDICINE”, which is hereby incorporated herein by reference in its entirety.

### FIELD

Generally, the aspects of the technology described herein relate to ultrasound data collection. Some aspects relate to ultrasound data collection using tele-medicine.

### BACKGROUND

Ultrasound devices may be used to perform diagnostic imaging and/or treatment, using sound waves with frequencies that are higher with respect to those audible to humans. Ultrasound imaging may be used to see internal soft tissue body structures, for example to find a source of disease or to exclude any pathology. When pulses of ultrasound are transmitted into tissue (e.g., by using a probe), sound waves are reflected off the tissue, with different tissues reflecting varying degrees of sound. These reflected sound waves may then be recorded and displayed as an ultrasound image to the operator. The strength (amplitude) of the sound signal and the time it takes for the wave to travel through the body provide information used to produce the ultrasound image. Many different types of images can be formed using ultrasound devices, including real-time images. For example, images can be generated that show two-dimensional cross-sections of tissue, blood flow, motion of tissue over time, the location of blood, the presence of specific molecules, the stiffness of tissue, or the anatomy of a three-dimensional region.

### BRIEF DESCRIPTION OF THE DRAWINGS

Various aspects and embodiments will be described with reference to the following exemplary and non-limiting figures. It should be appreciated that the figures are not necessarily drawn to scale. Items appearing in multiple figures are indicated by the same or a similar reference number in all the figures in which they appear.

FIG. 1 illustrates a schematic block diagram of an example ultrasound system upon which various aspects of the technology described herein may be practiced;

FIG. 2 illustrates an example operator graphical user interface (GUI) that may be displayed on an operator processing device, in accordance with certain embodiments described herein;

FIG. 3 illustrates an example instructor GUI that may be displayed on the instructor processing device, in accordance with certain embodiments described herein;

FIG. 4 illustrates the example instruction interface of the instructor GUI of FIG. 3, in accordance with certain embodiments described herein;

FIG. 5 illustrates the instruction interface of the instructor GUI of FIG. 3, in accordance with certain embodiments described herein;

FIGS. 6A and 6B illustrate example views of two faces of an ultrasound device, in accordance with certain embodiments described herein

FIG. 7 illustrates the instruction interface of the instructor GUI of FIG. 3, in accordance with certain embodiments described herein;

FIG. 8 illustrates the instruction interface of the instructor GUI of FIG. 3, in accordance with certain embodiments described herein;

FIG. 9 illustrates the instruction interface of the instructor GUI of FIG. 3, in accordance with certain embodiments described herein;

FIG. 10 illustrates the instruction interface of the instructor GUI of FIG. 3, in accordance with certain embodiments described herein;

FIG. 11 illustrates the instruction interface of the instructor GUI of FIG. 3, in accordance with certain embodiments described herein;

FIG. 12 illustrates the instruction interface of the instructor GUI of FIG. 3, in accordance with certain embodiments described herein;

FIG. 13 illustrates the instruction interface of the instructor GUI of FIG. 3, in accordance with certain embodiments described herein;

FIG. 14 illustrates another example instruction interface, in accordance with certain embodiments described herein;

FIG. 15 illustrates another example instruction interface, in accordance with certain embodiments described herein;

FIG. 16 illustrates another example translation interface, in accordance with certain embodiments described herein;

FIG. 17 illustrates another example translation interface, in accordance with certain embodiments described herein;

FIG. 18 illustrates an example of operation of the translation interface of FIG. 17, in accordance with certain embodiments described herein;

FIG. 19 illustrates another example translation interface, in accordance with certain embodiments described herein;

FIG. 20 illustrates an example of operation of the translation interface of FIG. 19, in accordance with certain embodiments described herein;

FIG. 21 illustrates another example translation interface, in accordance with certain embodiments described herein;

FIG. 22 illustrates an example process for displaying instructions for moving an ultrasound device on an operator processing device, in accordance with certain embodiments described herein.

FIG. 23 illustrates an example of an operator video, in accordance with certain embodiments described herein;

FIG. 24 illustrates an example of an operator video, in accordance with certain embodiments described herein;

FIG. 25 illustrates an example of an operator video, in accordance with certain embodiments described herein;

FIG. 26 illustrates an example process for displaying a directional indicator for translating an ultrasound device, in accordance with certain embodiments described herein;

FIG. 27 illustrates an example coordinate system for an ultrasound device, in accordance with certain embodiments described herein;

FIG. 28 illustrates an example process for displaying instructions for moving an ultrasound device on the instructor processing device, in accordance with certain embodiments described herein;

FIG. 29 illustrates an example of an operator video, in accordance with certain embodiments described herein;

FIG. 30 illustrates an example of an operator video, in accordance with certain embodiments described herein;

FIG. 31 illustrates an example of an operator video, in accordance with certain embodiments described herein;

FIG. 32 illustrates an example process for displaying an orientation indicator for an ultrasound device in an instruction interface, in accordance with certain embodiments described herein;

FIG. 33 illustrates an example process for displaying an orientation indicator for an ultrasound device in an operator video, in accordance with certain embodiments described herein;

FIG. 34 illustrates an example of the instructor GUI of FIG. 3, in accordance with certain embodiments described herein; and

FIG. 35 illustrates an example of the operator GUI of FIG. 2, in accordance with certain embodiments described herein.

### DETAILED DESCRIPTION

Conventional ultrasound systems are large, complex, and expensive systems that are typically only purchased by large medical facilities with significant financial resources. Recently, cheaper and less complex ultrasound devices have been introduced. Such imaging devices may include ultrasonic transducers monolithically integrated onto a single semiconductor die to form a monolithic ultrasound device. Aspects of such ultrasound-on-a chip devices are described in U.S. patent application Ser. No. 15/415,434 titled “UNIVERSAL ULTRASOUND DEVICE AND RELATED APPARATUS AND METHODS,” filed on Jan. 25, 2017 (and assigned to the assignee of the instant application), which is incorporated by reference herein in its entirety. The reduced cost and increased portability of these new ultrasound devices may make them significantly more accessible to the general public than conventional ultrasound devices.

The inventors have recognized and appreciated that although the reduced cost and increased portability of ultrasound devices makes them more accessible to the general populace, people who could make use of such devices have little to no training for how to use them. Ultrasound examinations often include the acquisition of ultrasound images that contain a view of a particular anatomical structure (e.g., an organ) of a subject. Acquisition of these ultrasound images typically requires considerable skill. For example, an ultrasound technician operating an ultrasound device may need to know where the anatomical structure to be imaged is located on the subject and further how to properly position the ultrasound device on the subject to capture a medically relevant ultrasound image of the anatomical structure. Holding the ultrasound device a few inches too high or too low on the subject may make the difference between capturing a medically relevant ultrasound image and capturing a medically irrelevant ultrasound image. As a result, non-expert operators of an ultrasound device may have considerable trouble capturing medically relevant ultrasound images of a subject. Common mistakes by these non-expert operators include capturing ultrasound images of the incorrect anatomical structure, capturing foreshortened (or truncated) ultrasound images of the correct anatomical structure, and failing to perform a complete study of the relevant anatomy (e.g., failing to scan all the anatomical regions of a particular protocol).

For example, a small clinic without a trained ultrasound technician on staff may purchase an ultrasound device to help diagnose patients. In this example, a nurse at the small clinic may be familiar with ultrasound technology and human physiology, but may know neither which anatomical views of a patient need to be imaged in order to identify medically-relevant information about the patient nor how to obtain such anatomical views using the ultrasound device. In another example, an ultrasound device may be issued to a patient by a physician for at-home use to monitor the patient's heart. In all likelihood, the patient understands neither human physiology nor how to image his or her own heart with the ultrasound device.

Accordingly, the inventors have developed tele-medicine technology, in which a human instructor, who may be remote from an operator of an ultrasound device, may instruct an operator how to move the ultrasound device in order to collect an ultrasound image. An operator may capture a video of the ultrasound device and the subject with a processing device (e.g., a smartphone or tablet) and the video, in addition to ultrasound images collected by the ultrasound device, may be transmitted to the instructor to view and use in providing instructions for moving the ultrasound device. (Additionally, the instructor may transmit audio to the operator's processing device and cause the operator processing device to configure the ultrasound device with imaging settings and parameter values.) However, the inventors have recognized that providing such instructions may be difficult. For example, a verbal instruction to move an ultrasound device “up” may be ambiguous in that it could be unclear whether “up” is relative to the operator's perspective, relative to the subject's anatomy, or perhaps relative to the ultrasound device itself.

Accordingly, the inventors have developed technology in which directional indicators (e.g., arrows) may be superimposed on video collected by the operator's processing device. However, the inventors have recognized that even when directional indicators are superimposed on video of the operator's environment, the meaning of such directional indicators may still be ambiguous. For example, when presented with a two-dimensional arrow superimposed on a video, an operator may not clearly understand how to follow this instruction in a three-dimensional context. The inventors have therefore recognized that it may be helpful for an instruction such as an arrow to be displayed in video such that the arrow appears relative to the location and orientation of the ultrasound device. In other words, the arrow may appear in the video to be part of the three-dimensional environment of the ultrasound device. This may help the instruction to be more useful and clearer in meaning. The inventors have also recognized that verbal instructions such as “up” may be lacking, as an instructor may wish the operator to move the ultrasound device in a direction that cannot be conveyed with words like “up” and “down.” Accordingly, the inventors have developed graphical user interfaces that may provide an instructor with a wide and flexible range of instruction options. The graphical user interfaces may include indicators of the orientation of the ultrasound device in the video of the operator's environment to assist the instructor in selecting instructions.

It should be appreciated that the embodiments described herein may be implemented in any number of ways. Examples of specific implementations are provided below for illustrative purposes only. It should be appreciated that these embodiments and the features/capabilities provided may be used individually, all together, or in any combination of two or more, as aspects of the technology described herein are not limited in this respect.

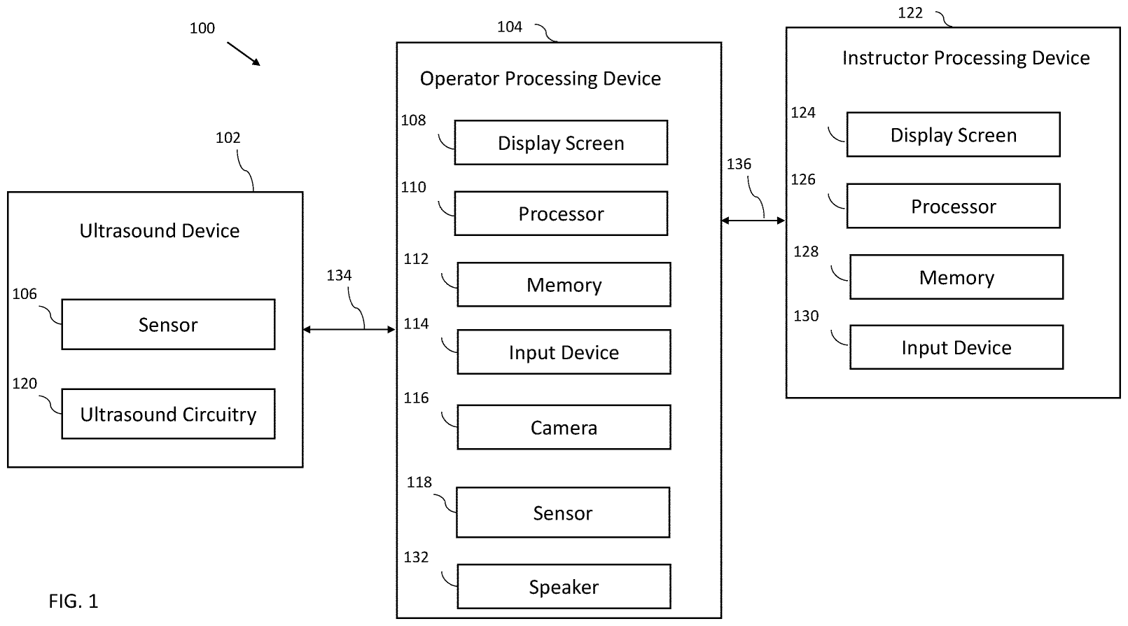

FIG. 1 illustrates a schematic block diagram of an example ultrasound system 100 upon which various aspects of the technology described herein may be practiced. The ultrasound system 100 includes an ultrasound device 102, an operator processing device 104, and an instructor processing device 122. The operator processing device 104 may be associated with an operator of the ultrasound device 102 and the instructor processing device 122 may be associated with an instructor who provides instructions to the operator for moving the ultrasound device 102. The operator processing device 104 and the instructor processing device 122 may be remote from each other.

The ultrasound device 102 includes a sensor 106 and ultrasound circuitry 120. The operator processing device 104 includes a camera 116, a display screen 108, a processor 110, a memory 112, an input device 114, a sensor 118, and a speaker 132. The instructor processing device 122 includes a display screen 124, a processor 126, a memory 128, and an input device 130. The operator processing device 104 and the ultrasound device 102 are in communication over a communication link 134, which may be wired, such as a lightning connector or a mini-USB connector, and/or wireless, such as a link using BLUETOOTH, ZIGBEE, and/or WiFi wireless protocols. The operator processing device 104 and the instructor processing device 122 are in communication over a communication link 136, which may be wired, such as a lightning connector or a mini-USB connector, and/or wireless, such as a link using BLUETOOTH, ZIGBEE, and/or WiFi wireless protocols.

The ultrasound device 102 may be configured to generate ultrasound data that may be employed to generate an ultrasound image. In some embodiments, the ultrasound circuitry 120 includes a transmitter that transmits a signal to a transmit beamformer which in turn drives transducer elements within a transducer array to emit pulsed ultrasonic signals into a structure, such as a patient. The pulsed ultrasonic signals may be back-scattered from structures in the body, such as blood cells or muscular tissue, to produce echoes that return to the transducer elements. These echoes may then be converted into electrical signals by the transducer elements and the electrical signals are received by a receiver. The electrical signals representing the received echoes may be sent to a receive beamformer that outputs ultrasound data. The transducer elements, which may also be part of the ultrasound circuitry 120, may include one or more ultrasonic transducers monolithically integrated onto a single semiconductor die. The ultrasonic transducers may include, for example, one or more capacitive micromachined ultrasonic transducers (CMUTs), one or more CMOS (complementary metal-oxide-semiconductor) ultrasonic transducers (CUTs), one or more piezoelectric micromachined ultrasonic transducers (PMUTs), and/or one or more other suitable ultrasonic transducer cells. In some embodiments, the ultrasonic transducers may be formed the same chip as other electronic components in the ultrasound circuitry 120 (e.g., transmit circuitry, receive circuitry, control circuitry, power management circuitry, and processing circuitry) to form a monolithic ultrasound device. The ultrasound device 102 may transmit ultrasound data and/or ultrasound images to the operator processing device 104 over the communication link 134.

The sensor 106 may be configured to generate motion and/or orientation data regarding the ultrasound device 102. For example, the sensor 106 may be configured to generate data regarding acceleration of the ultrasound device 102, data regarding angular velocity of the ultrasound device 102, and/or data regarding magnetic force acting on the ultrasound device 102 (which, due to the magnetic field of the earth, may be indicative of orientation relative to the earth). The sensor 106 may include an accelerometer, a gyroscope, and/or a magnetometer. Depending on the sensors present in the sensor 106, the motion and orientation data generated by the sensor 106 may describe three degrees of freedom, six degrees of freedom, or nine degrees of freedom for the ultrasound device 102. For example, the sensor 106 may include an accelerometer, a gyroscope, and/or magnetometer. Each of these types of sensors may describe three degrees of freedom. If the sensor 106 includes one of these sensors, the sensor 106 may describe three degrees of freedom. If the sensor 106 includes two of these sensors, the sensor 106 may describe two degrees of freedom. If the sensor 106 includes three of these sensors, the sensor 106 may describe nine degrees of freedom. The ultrasound device 102 may transmit data to the operator processing device 104 over the communication link 134.

Referring now to the operator processing device 104, the processor 110 may include specially-programmed and/or special-purpose hardware such as an application-specific integrated circuit (ASIC). For example, the processor 110 may include one or more graphics processing units (GPUs) and/or one or more tensor processing units (TPUs). TPUs may be ASICs specifically designed for machine learning (e.g., deep learning). The TPUs may be employed to, for example, accelerate the inference phase of a neural network. The operator processing device 104 may be configured to process the ultrasound data received from the ultrasound device 102 to generate ultrasound images for display on the display screen 108. The processing may be performed by, for example, the processor 110. The processor 110 may also be adapted to control the acquisition of ultrasound data with the ultrasound device 102. The ultrasound data may be processed in real-time during a scanning session as the echo signals are received. In some embodiments, the displayed ultrasound image may be updated a rate of at least 5 Hz, at least 10 Hz, at least 20 Hz, at a rate between 5 and 60 Hz, at a rate of more than 20 Hz. For example, ultrasound data may be acquired even as images are being generated based on previously acquired data and while a live ultrasound image is being displayed. As additional ultrasound data is acquired, additional frames or images generated from more-recently acquired ultrasound data are sequentially displayed. Additionally, or alternatively, the ultrasound data may be stored temporarily in a buffer during a scanning session and processed in less than real-time.

The operator processing device 104 may be configured to perform certain of the processes described herein using the processor 110 (e.g., one or more computer hardware processors) and one or more articles of manufacture that include non-transitory computer-readable storage media such as the memory 112. The processor 110 may control writing data to and reading data from the memory 112 in any suitable manner. To perform certain of the processes described herein, the processor 110 may execute one or more processor-executable instructions stored in one or more non-transitory computer-readable storage media (e.g., the memory 112), which may serve as non-transitory computer-readable storage media storing processor-executable instructions for execution by the processor 110. The camera 116 may be configured to detect light (e.g., visible light) to form an image or a video. The display screen 108 may be configured to display images and/or videos, and may be, for example, a liquid crystal display (LCD), a plasma display, and/or an organic light emitting diode (OLED) display on the operator processing device 104. The input device 114 may include one or more devices capable of receiving input from an operator and transmitting the input to the processor 110. For example, the input device 114 may include a keyboard, a mouse, a microphone, touch-enabled sensors on the display screen 108, and/or a microphone. The sensor 118 may be configured to generate motion and/or orientation data regarding the operator processing device 104. Further description of sensors may be found with reference to the sensor 106. The speaker 132 may be configured to output audio from the operator processing device 104. The display screen 108, the input device 114, the camera 116, the speaker 106, and the sensor 118 may be communicatively coupled to the processor 110 and/or under the control of the processor 110.

It should be appreciated that the operator processing device 104 may be implemented in any of a variety of ways. For example, the operator processing device 104 may be implemented as a handheld device such as a mobile smartphone or a tablet. Thereby, an operator of the ultrasound device 102 may be able to operate the ultrasound device 102 with one hand and hold the operator processing device 104 with another hand. Or, a holder may hold the operator processing device 104 in place (e.g., with a clamp). In other examples, the operator processing device 104 may be implemented as a portable device that is not a handheld device, such as a laptop. In yet other examples, the operator processing device 104 may be implemented as a stationary device such as a desktop computer.

Referring now to the instructor processing device 122, further description of the display screen 124, the processor 126, the memory 128, and the input device 130 may be found with reference to the display screen 108, the processor 110, the memory 112, and the input device 114, respectively. It should be appreciated that the instructor processing device 122 may be implemented in any of a variety of ways. For example, the instructor processing device 122 may be implemented as a handheld device such as a mobile smartphone or a tablet, as a portable device that is not a handheld device, such as a laptop, or as a stationary device such as a desktop computer. For further description of ultrasound devices and systems, see U.S. patent application Ser. No. 15/415,434 titled “UNIVERSAL ULTRASOUND DEVICE AND RELATED APPARATUS AND METHODS,” filed on Jan. 25, 2017 (and assigned to the assignee of the instant application). FIG. 1 should be understood to be non-limiting. For example, the ultrasound device 102, the operator processing device 104, and the instructor processing device 122 may include fewer or more components than shown.

### Operator and Instructor Graphical User Interfaces

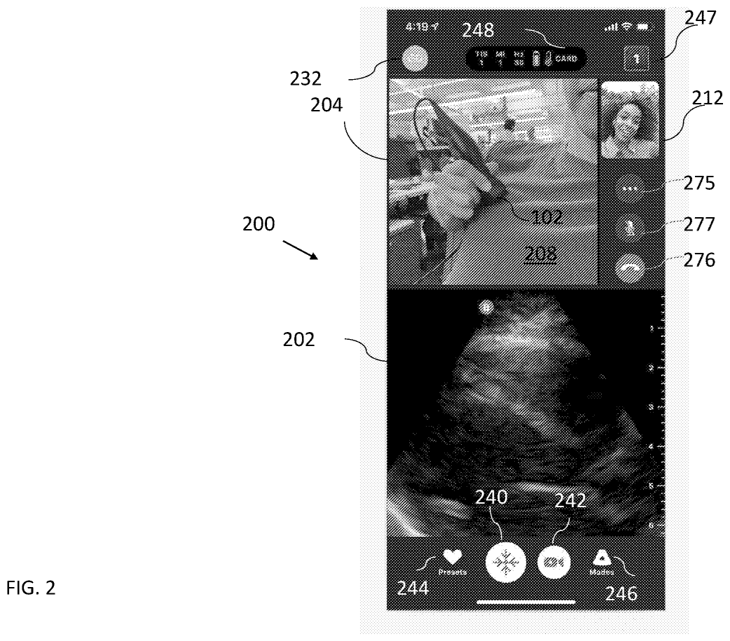

FIG. 2 illustrates an example operator graphical user interface (GUI) 200 that may be displayed on the operator processing device 104, in accordance with certain embodiments described herein. The operator GUI 200 includes an ultrasound image 202 and an operator video 204.

The ultrasound image 202 may be generated from ultrasound data collected by the ultrasound device 102. In some embodiments, the ultrasound device 102 may transmit raw acoustical data or data generated from the raw acoustical data (e.g., scan lines) to the operator processing device 104, and the operator processing device 104 may generate the ultrasound image 202 and transmit the ultrasound image 202 to the instructor processing device 122. In some embodiments, the ultrasound device 102 may generate the ultrasound image 202 from raw acoustical data and transmit the ultrasound image 202 to the operator processing device 104, and the operator processing device 104 may transmit the ultrasound image 202 to the instructor processing device 122 for display. In some embodiments, as the ultrasound device 102 collects more ultrasound data, the operator processing device 104 may update the ultrasound image 202 with a new ultrasound image 202 generated from the new ultrasound data.

The operator video 204 depicts a subject 208 being imaged (where the subject 208 may be the same as the operator) and the ultrasound device 102. In some embodiments, the operator video 204 may be captured by a front-facing camera (e.g., the camera 116) on the operator processing device 104. Such embodiments may be more appropriate when the operator is the same as the subject 208 being imaged. However, in some embodiments, the operator video 204 may be captured by a rear-facing camera (e.g., the camera 116) on the operator processing device 104. Such embodiments may be more appropriate when the operator is different from the subject 208 being imaged. In either case, the operator or a holder (e.g., a stand having a clamp for clamping the operator processing device 104 in place) may hold the operator processing device 104 such that the ultrasound device 102 and portions of the subject 208 adjacent to the ultrasound device 102 are within view of the camera 116. Or, in either case, the operator processing device 104 may be a stationary device such as a laptop, and the subject 208 and the ultrasound device 102 may be positioned to be in view of the camera 116 of the operator processing device 104. In some embodiments, the operator processing device 104 may transmit the operator video 204 to the instructor processing device 122 for display.

In some embodiments, such as that of FIG. 2, when the operator processing device 104 captures the operator video 204 using a front-facing camera (e.g., the camera 116), the operator processing device 104 may horizontally flip the operator video 204 as captured by the front-facing camera (e.g., the camera 116) prior to displaying the video as the operator video 204 in the operator GUI 200. As discussed above, using a front-facing camera (e.g., the camera 116) may be more appropriate when the operator is also the subject 208 being imaged, and thus in such embodiments, the operator may be viewing a video of himself/herself in the operator video 204. Flipping the operator video 204 horizontally may make the operator video 204 appear like a reflection of the operator in a mirror, which may be a familiar manner for the operator to view a video of himself/herself. However, as will be described further below, the operator video 204 may not be flipped horizontally when displayed on the instructor processing device 122. Additionally, when the operator processing device 104 captures the operator video 204 using a rear-facing camera (e.g., the camera 116), the operator processing device 104 may not flip the operator video 204 horizontally, as such embodiments may be more appropriate when the operator is not the subject 208 being imaged, and thus the operator video 204 appearing like a mirror reflection may not be helpful.

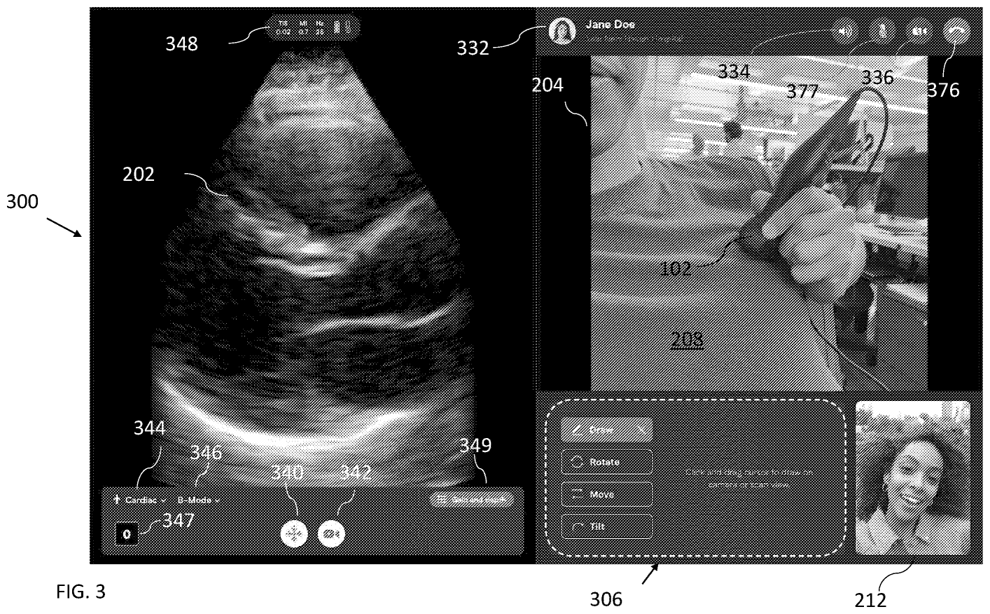

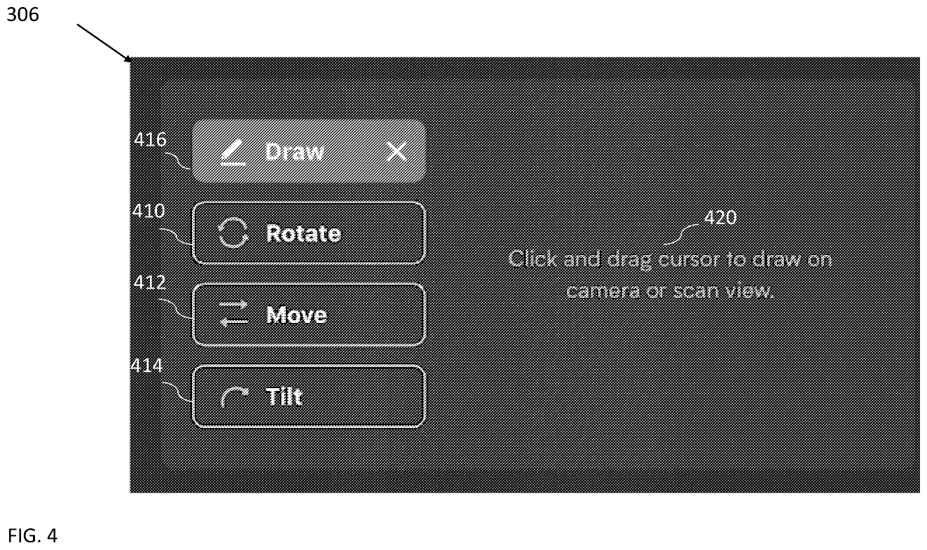

FIG. 3 illustrates an example instructor GUI 300 that may be displayed on the instructor processing device 122, in accordance with certain embodiments described herein. The instructor GUI 300 includes the ultrasound image 202, the operator video 204, and an instruction interface 306. Further description of the instruction interface 306 may be found with reference to FIGS. 4-13.

As described above, in some embodiments such as those of FIGS. 2 and 3, when the operator processing device 104 captures the operator video 204 using a front-facing camera (e.g., the camera 116), the operator processing device 104 may flip the operator video 204 as captured by the front-facing camera (e.g., the camera 116) horizontally prior to displaying the video as the operator video 204 in the operator GUI 200. However, the operator video 204 may not be flipped horizontally when displayed on the instructor GUI 300. Thus, the operator video 204 in the operator GUI 200 and the operator 204 in the instructor GUI 300 may be flipped horizontally from one another.

### Graphical User Interfaces for Selecting Instructions

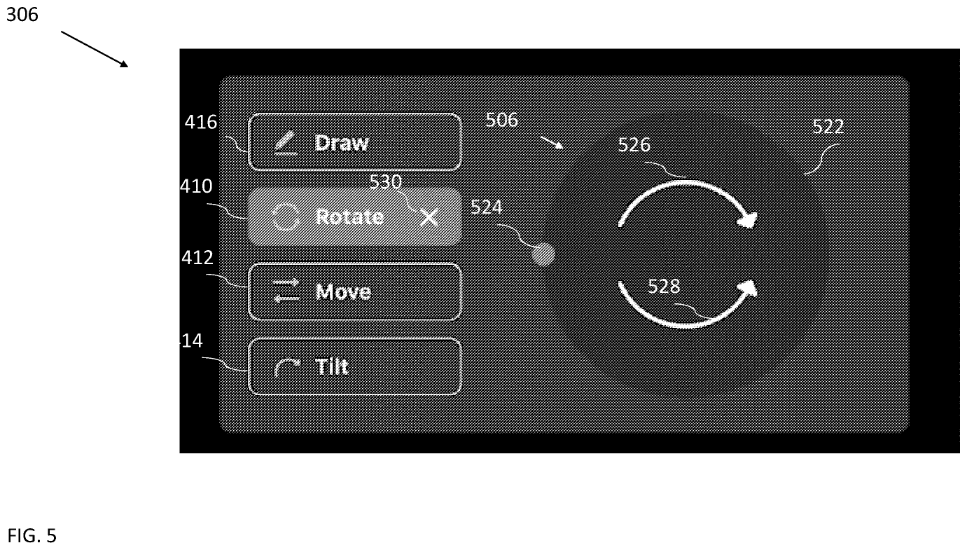

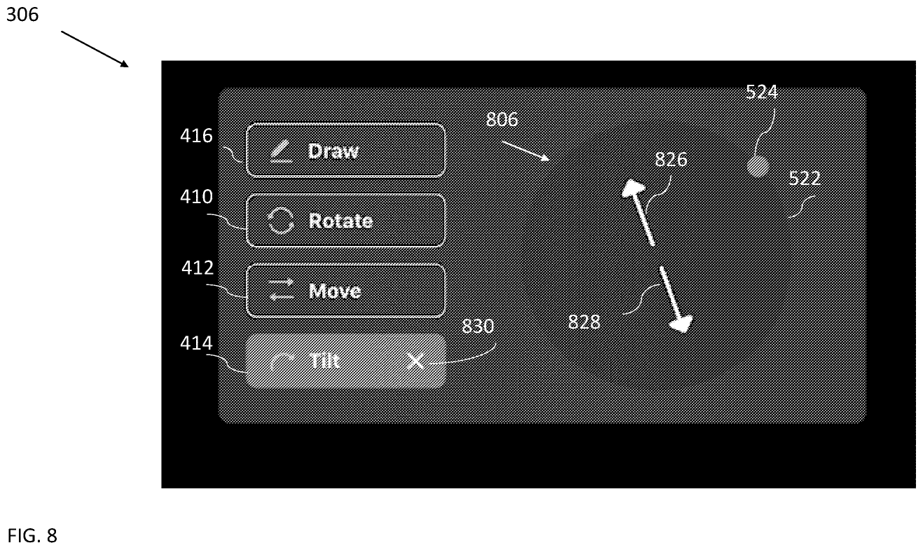

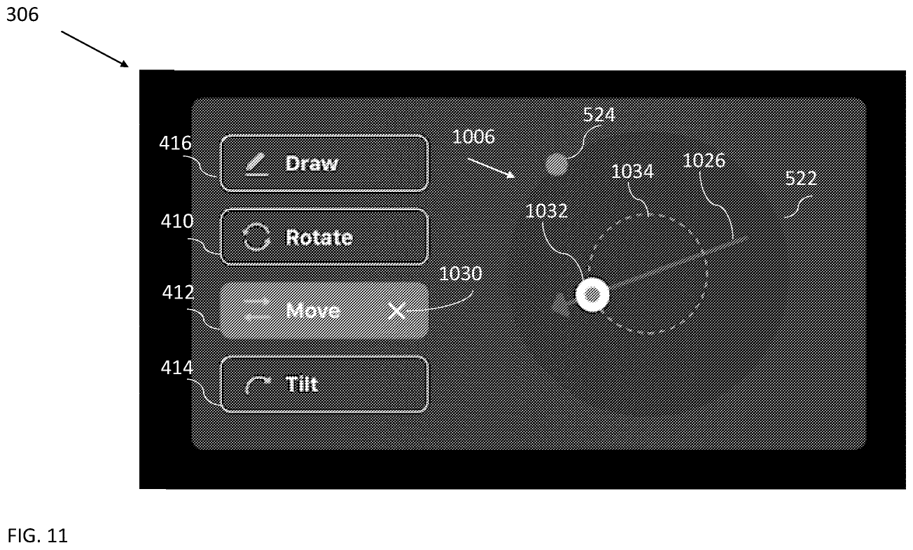

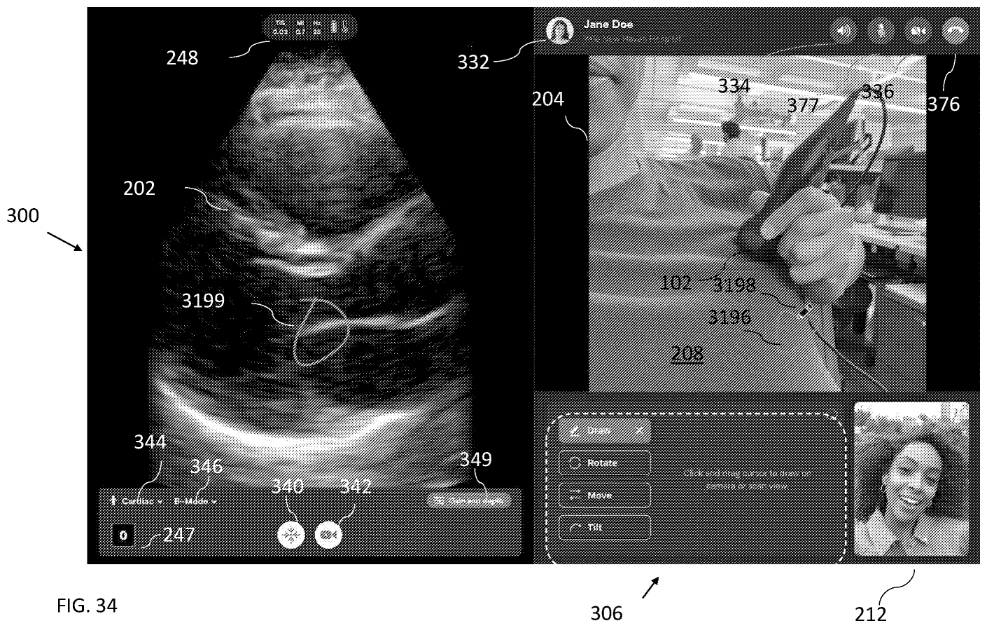

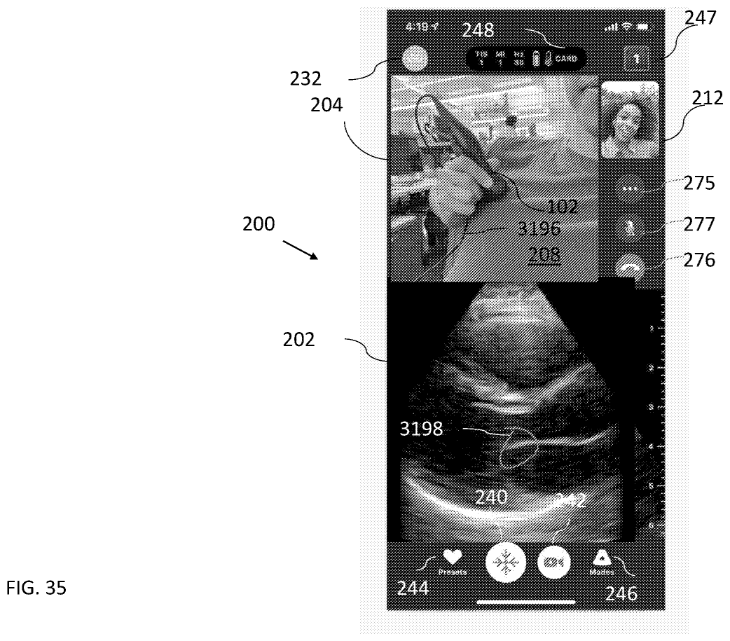

FIG. 4 illustrates the example instruction interface 306 of the instructor GUI 300, in accordance with certain embodiments described herein. The instruction interface 306 in FIG. 4 includes a rotate option 410, a tilt option 414, a move option 412, a draw option 416, and text 420. The text 420 indicates that the instructor should choose one of the displayed options. In response to a selection from the instructor of the rotate option 410, the instruction interface 306 may display the rotation interface 506 of FIG. 5. In response to a selection from the instructor of the tilt option 414, the instruction interface 306 may display the tilt interface 806 of FIG. 8. In response to a selection from the instructor of the move option 412, the instruction interface 306 may display the translation interface 1006 of FIG. 11. In response to a selection from the instructor of the draw option 416, the instructor GUI 300 may permit drawing on the ultrasound image 202 and/or the operator video 204, as will be described further with reference to FIGS. 34-35. In FIG. 4, the draw option 416 is highlighted. In some embodiments, FIG. 4 may illustrate the instruction interface 306 in a default state. In some embodiments, instead of selecting the rotate option 410, the tilt option 414, or the move option 412 to show the rotation interface 506, the tilt interface 806, or the translation interface 1006, respectively, the rotation interface 506, the tilt interface 806, and the translation interface 1006 may be displayed simultaneously. In some embodiments, rather than displaying the draw option 416, the draw state (in which the instructor GUI 300 may permit drawing on the ultrasound image 202 and/or the operator video 204) may be entered whenever none of the rotate option 410, move option 412, or tilt option 414 are selected.

FIG. 5 illustrates the instruction interface 306 of the instructor GUI 300, in accordance with certain embodiments described herein. In FIG. 5, the instruction interface 306 displays a rotation interface 506. The instruction interface 306 may display the rotation interface 506 in response to a selection of the rotate option 410. Furthermore, in response to selection of the rotate option 410, the rotate option 410 may be highlighted (e.g., with a change of color) and an exit option 530 may be displayed in the rotate option 410, as illustrated. In response to a selection of the exit option 530, the instruction interface 306 may display a default state of the instruction interface 306 (e.g., the state in FIG. 4).

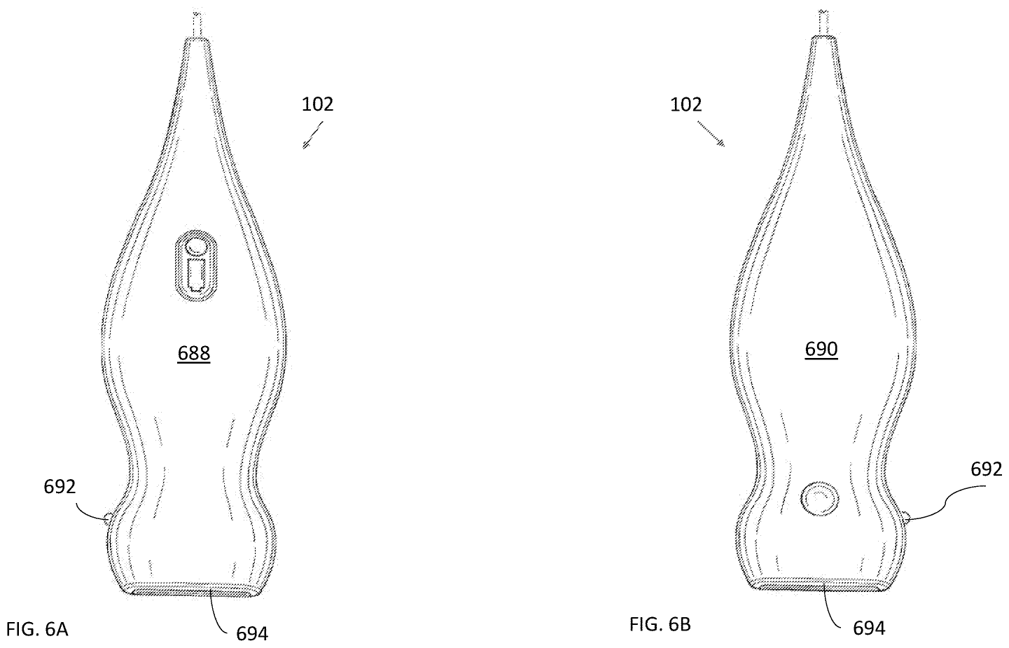

The rotation interface 506 includes a circle 522, an orientation indicator 524, a clockwise rotation option 526, and a counterclockwise rotation option 528. The orientation indicator 524 may indicate the orientation of the ultrasound device 102 relative to the operator processing device 104. In particular, the position of the orientation indicator 524 around the circle 522 may be based on the pose of a marker 692 (illustrated in FIGS. 6A and 6B) on the ultrasound device 102 relative to the operator processing device 104.

FIGS. 6A and 6B illustrate example views of two faces 688 and 690 of the ultrasound device 102, in accordance with certain embodiments described herein. The ultrasound device 102 includes a marker 692 between the two faces 688 and 690 and an ultrasound transducer array 694. The marker 692 may serve as an indication of the orientation of the ultrasound device 102. For example, if from an operator's perspective the ultrasound transducer array 694 is facing downwards and the marker 692 is on the left of the ultrasound device 102, then the operator may know that the face 688 is facing the operator. If from the operator's perspective the ultrasound transducer array 694 is facing downwards and the marker 692 is on the right of the ultrasound device 102, then the operator may know that the face 690 is facing the operator.

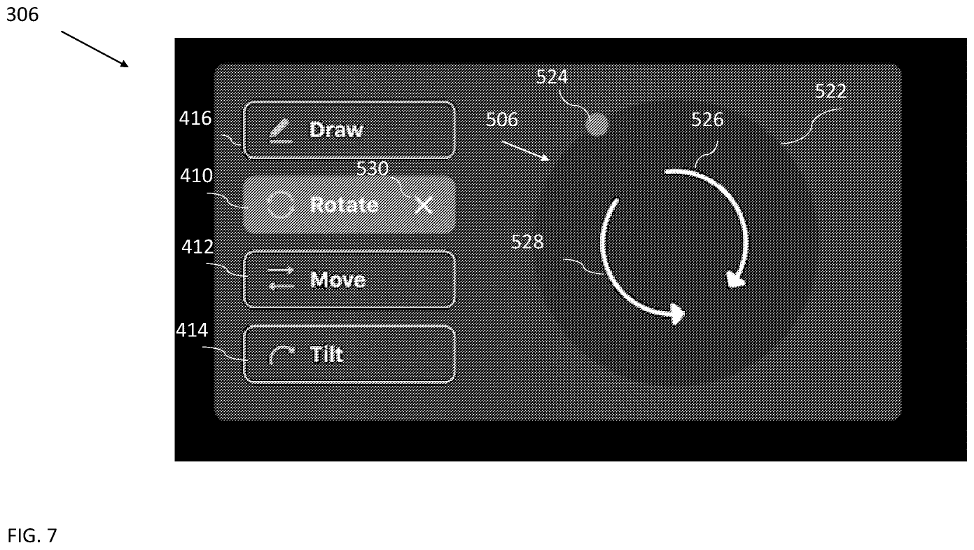

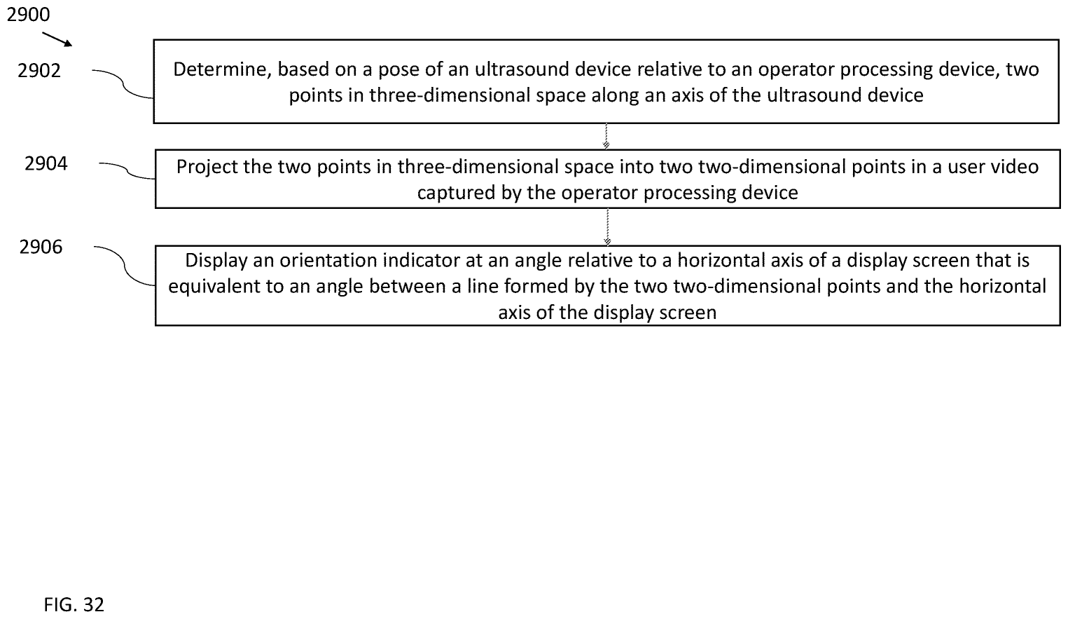

Referring back to FIG. 5, generally, the orientation indicator 524 may illustrate the direction the ultrasound device 102's marker 692 is pointing relative to the operator video 204 (in other words, relative to the operator processing device 104, and more particularly, the camera 116 on the operator processing device 104). The orientation indicator 524 may indicate two-dimensionally a three-dimensional pose of the marker 692. Thus, as the pose of the marker 692 on the ultrasound device 102 relative to the operator processing device 104 changes due to movement of the ultrasound device 102 relative to the operator processing device 104, the position of the orientation indicator 524 around the circle 522 may change. As an example, FIG. 7 illustrates the instruction interface 306 of the instructor GUI 300, where the instruction interface 306 includes the rotation interface 506 with the orientation indicator 524 at another position around the circle 522, in accordance with certain embodiments described herein. Further description of determining the position of the orientation indicator 524 around the circle 522 may be found with reference to FIG. 32. In FIG. 7, the clockwise rotation option 526 and the counterclockwise rotation option 528 have also rotated about the circle 522 along with the orientation indicator 524, although in other embodiments the clockwise rotation option 526 and the counterclockwise rotation option 528 may not move even as the orientation indicator 524 moves.

In FIG. 5, the clockwise rotation option 526 and the counterclockwise rotation option 528 are arrows. In some embodiments, in response to a hover over the clockwise rotation option 526 or the counterclockwise rotation option 528, the rotation interface 506 may display that option (i.e., the arrow) in a different color. In some embodiments, in response to a selection of the clockwise rotation option 526 or the counterclockwise rotation option 528, the rotation interface 506 may display that option in another different color. Additionally, the instructor processing device 122 may output to the operator processing device 104 either a clockwise rotation or a counterclockwise rotation instruction, corresponding to the selected option.

FIG. 8 illustrates the instruction interface 306 of the instructor GUI 300, in accordance with certain embodiments described herein. In FIG. 8, the instruction interface 306 displays a tilt interface 806. The instruction interface 306 may display the rotation interface 806 in response to a selection of the tilt option 414. Furthermore, in response to selection of the tilt option 414, the tilt option 414 may be highlighted (e.g., with a change of color) and an exit option 830 may be displayed in the tilt option 414, as illustrated. In response to a selection of the exit option 830, the instruction interface 306 may display a default state of the instruction interface 306 (e.g., the state in FIG. 4). The tilt interface 806 includes the circle 522, the orientation indicator 524, a tilt option 826, and a tilt option 828. In FIG. 8, the tilt option 826 and the tilt option 828 are arrows.

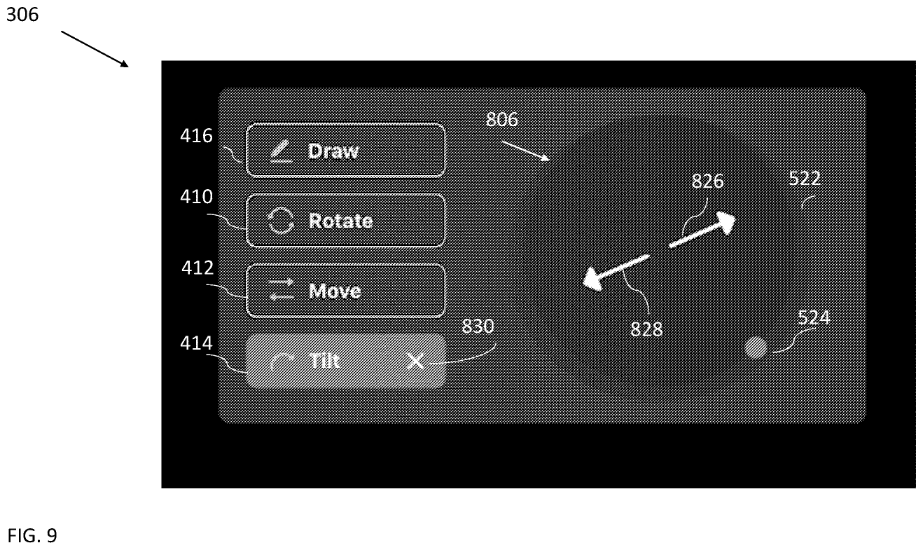

As described above, the orientation indicator 524 may indicate the orientation of the ultrasound device 102 relative to the operator processing device 104, and thus, as the pose of the marker 692 on the ultrasound device 102 relative to the operator processing device 104 changes due to movement of the ultrasound device 102 relative to the operator processing device 104, the position of the orientation indicator 524 around the circle 522 may change. As an example, FIG. 9 illustrates the instruction interface 306 of the instructor GUI 300, where the instruction interface 306 includes the tilt interface 806 with the orientation indicator 524 at another position around the circle 522, in accordance with certain embodiments described herein. In FIG. 9, the tilt option 826 and the tilt option 828 have also rotated about the circle 522 along with the orientation indicator 524.

The position of the orientation indicator 524 around the circle 522 may assist the instructor in selecting the tilt option 866 or the tilt option 828, because the orientation indicator 524 may indicate to which face of the ultrasound device 102 each of the tilt options 826 and 828 correspond. For example, in FIG. 8, the orientation indicator 524 is on the right side of the circle 522, and if the ultrasound device 102 is pointing downwards, then the face 690 of the ultrasound device 102 may be facing towards the operator and the face 688 of the ultrasound device 102 may be facing away from the operator. Thus, the tilt option 826 may correspond to an instruction to tilt the face 688 of the ultrasound device 102 towards the subject 208 and the tilt option 828 may correspond to an instruction to tilt the face 690 of the ultrasound device 102 towards the subject 208. In some embodiments, in response to a hover over the tilt option 826 or the tilt option 828, the tilt interface 806 may display that option (i.e., the arrow) in a different color. In some embodiments, in response to a selection of the tilt option 826 or the tilt option 828, the tilt interface 806 may display that option (i.e., the arrow) in another different color. Additionally, the instructor processing device 122 may output to the operator processing device 104 either an instruction to tilt the face 688 of the ultrasound device 102 towards the subject 208 or to tilt the face 690 of the ultrasound device 102 towards the subject 208, corresponding to the selected option.

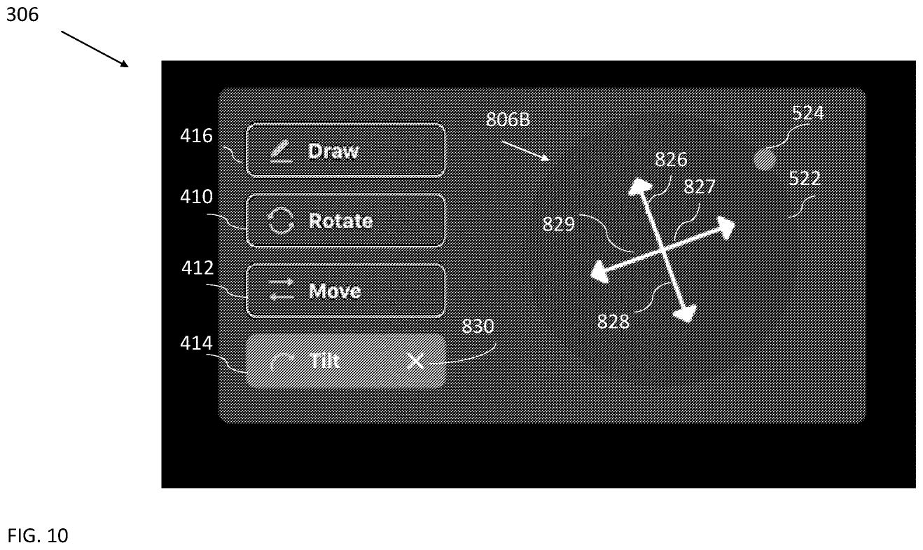

FIG. 10 illustrates the instruction interface 306 of the instructor GUI 300, in accordance with certain embodiments described herein. In FIG. 10, the instruction interface 306 displays a tilt interface 806B. The tilt interface 806B is the same as the tilt interface 806, except that the tilt interface 806B additionally includes a tilt option 827 and a tilt option 829. Thus, each of the tilt options 826-829 corresponds to instructions to tilt one of the four faces of the ultrasound device 102.

FIG. 11 illustrates the instruction interface 306 of the instructor GUI 300, in accordance with certain embodiments described herein. In FIG. 11, the instruction interface 306 displays a translation interface 1006. The instruction interface 306 may display the translation interface 1006 in response to a selection of the move option 412. Furthermore, in response to selection of the move option 412, the move option 412 may be highlighted (e.g., with a change of color) and an exit option 1030 may be displayed in the move option 412, as illustrated. In response to a selection of the exit option 1030, the instruction interface 306 may display a default state of the instruction interface 306 (e.g., the state in FIG. 4).

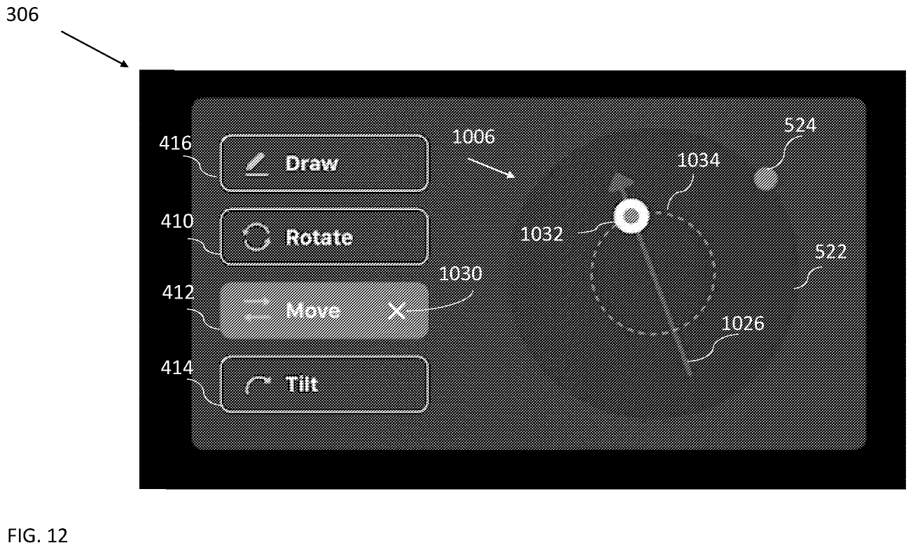

The orientation indicator 524 may indicate the orientation of the ultrasound device 102 relative to the operator processing device 104. In particular, the position of the orientation indicator 524 around the circle 522 may be based on the pose of the marker 692 on the ultrasound device 102 relative to the operator processing device 104. Generally, the orientation indicator 524 may illustrate the direction the ultrasound device 102's marker 692 is pointing relative to the operator video 204. The orientation indicator 524 may indicate two-dimensionally a three-dimensional pose of the marker 692. Thus, as the pose of the marker 692 on the ultrasound device 102 relative to the operator processing device 104 changes due to movement of the ultrasound device 102 relative to the operator processing device 104, the position of the orientation indicator 524 around the circle 522 may change. As an example, FIG. 12 illustrates the instruction interface 306 of the instructor GUI 300, where the instruction interface 306 includes the translation interface 1006 with the orientation indicator 524 at another position around the circle 522, in accordance with certain embodiments described herein. Further description of determining the position of the orientation indicator 524 around the circle 522 may be found with reference to FIG. 32. In FIG. 12, the arrow 1026 and the cursor 1032 have also rotated about the circle 522 along with the orientation indicator 524, although in other embodiments the arrow 1026 and the cursor 1032 may not move even as the orientation indicator 524 moves.

In some embodiments, in response to a hover over the cursor 1032, the arrow 1026 and the cursor 1032 may stop moving even as the orientation indicator 524 moves. In some embodiments, in response to a dragging movement (e.g., dragging a finger or stylus or holding down a mouse button and moving the mouse) beginning on or near the cursor 1032, the cursor 1032 and the arrow 1026 may rotate about the circle 1034 based on the dragging movement. For example, in response to a dragging movement moving clockwise about the circle 1034, the cursor 1032 and the arrow 1026 may rotate clockwise about the circle 1034. In some embodiments, in response to cessation of the dragging movement (e.g., releasing a finger or releasing a mouse button), the cursor 1032 and the arrow 1026 may cease to move, and the translation interface 1006 may display the arrow 1026 in a different color. This may correspond to selection of the particular angle of the arrow 1026 with respect to the horizontal axis of the circle 1034. The instructor processing device 122 may output to the operator processing device 104 the selected angle for translation.

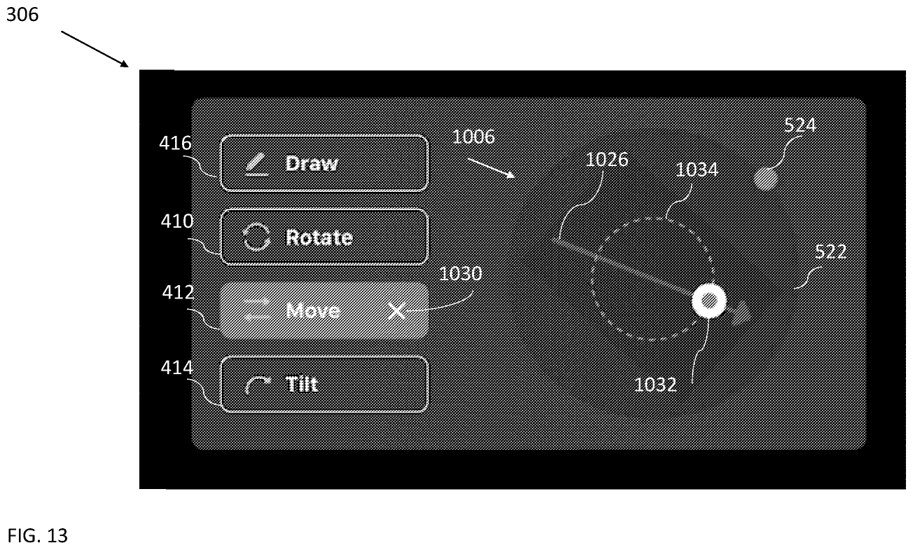

As an example, FIG. 13 illustrates the instruction interface 306 of the instructor GUI 300, where the instruction interface 306 includes the translation interface 1006 after the cursor 1032 and the arrow 1026 have rotated about the circle 1034 (from their positions in FIG. 12) in response to a dragging movement beginning on or near the cursor 1032, in accordance with certain embodiments described herein. It should be appreciated that the movement of the cursor 1032 and the arrow 1026 from FIG. 12 to FIG. 13 is due to a dragging movement beginning on or near the cursor 1032, while the movement of the cursor 1032 and the arrow 1026 from FIG. 11 to FIG. 12 is due to movement of the ultrasound device 102 relative to the operator processing device 104. Thus, the orientation indicator 524, which may also move in response to movement of the ultrasound device 102 relative to the operator processing device 104, has moved from FIG. 11 to FIG. 12 but not from FIG. 12 to FIG. 13.

The position of the orientation indicator 524 around the circle 522 may assist the instructor in selecting an instruction from the translation interface 1006. For example, if an instructor, viewing the operator video 204, wishes to provide an instruction to the operator to move the ultrasound device 102 in the direction that the marker 692 on the ultrasound device 102 is pointing, then the instructor may rotate the arrow 1026 to point towards orientation indicator 524. If an instructor, viewing the operator video 204, wishes to provide an instruction to the operator to move the ultrasound device 102 opposite the direction that the marker 692 on the ultrasound device 102 is pointing, then the instructor may rotate the arrow 1026 to point away from the orientation indicator 524.

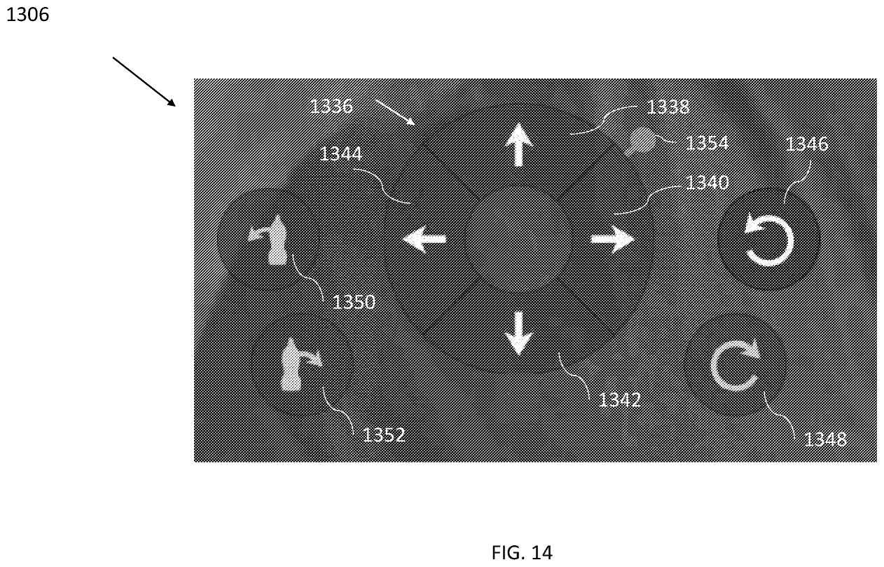

FIG. 14 illustrates another example instruction interface 1306, in accordance with certain embodiments described herein. The instruction interface 1306 includes a translation interface 1336. The translation interface 1336 is circular and includes an up option 1338, a right option 1340, a down option 1342, and a left option 1344. The instruction interface 1306 further includes a counterclockwise option 1346, a clockwise option 1348, a tilt option 1350, a tilt option 1352, and an orientation indicator 1354.

As with the orientation indicator 524, the orientation indicator 1343 indicates the orientation of the ultrasound device 102 relative to the operator processing device 104. In particular, the position of the orientation indicator 524 around the circle of the translation interface 1336 may be based on the pose of the marker 692 on the ultrasound device 102 relative to the operator processing device 104. Generally, the orientation indicator 524 may illustrate the direction the ultrasound device 102's marker 692 is pointing relative to the operator video 204. The orientation indicator 524 may indicate two-dimensionally a three-dimensional pose of the marker 692. Thus, as the pose of the marker 692 on the ultrasound device 102 relative to the operator processing device 104 changes due to movement of the ultrasound device 102 relative to the operator processing device 104, the position of the orientation indicator 524 around the circle of the translation interface 1336 may change.

In some embodiments, in response to receiving a selection of the right option 1340, the up option 1338, the left option 1344, or the down option 1342, the instructor processing device 122 may output to the operator processing device 104 an angle for translation corresponding to the selected option (e.g., 0, 90, 180, or 270 degrees, respectively). In some embodiments, in response to receiving a selection of the counterclockwise option 1346 or the clockwise option 1348, the instructor processing device 122 may output to the operator processing device 104 either a counterclockwise rotation or a clockwise rotation instruction, corresponding to the selected option. In some embodiments, in response to receiving a selection of the tilt option 1350 or the tilt option 1352, the instructor processing device 122 may output to the operator processing device 104 an instruction to tilt one of the faces 688 or 690 of the ultrasound device 102 towards the subject 208, corresponding to the selected option. In other words, in some embodiments, the tilt option 1350 may correspond to tilting the face 688 of the ultrasound device 102 towards the subject 208 and the tilt option 1352 may correspond to tilting the face 690 of the ultrasound device 102 towards the subject 208, or vice versa. However, in some embodiments, the instructions outputted in response to selection of the one of the tilt options 1350 and 1352 may depend on the location of the orientation indicator 1354. For example, if the orientation indicator 1354 is on the right side of the circle of the translation interface 1336, then the tilt option 1350 may correspond to tilting the face 690 of the ultrasound device 102 towards the subject 208 and the tilt option 1352 may correspond to tilting the face 688 of the ultrasound device 102 towards the subject. If the orientation indicator 1354 is on the left side of the circle of the translation interface 1336, then the tilt option 1350 may correspond to tilting the face 688 of the ultrasound device 102 towards the subject 208 and the tilt option 1352 may correspond to tilting the face 690 of the ultrasound device 102 towards the subject.

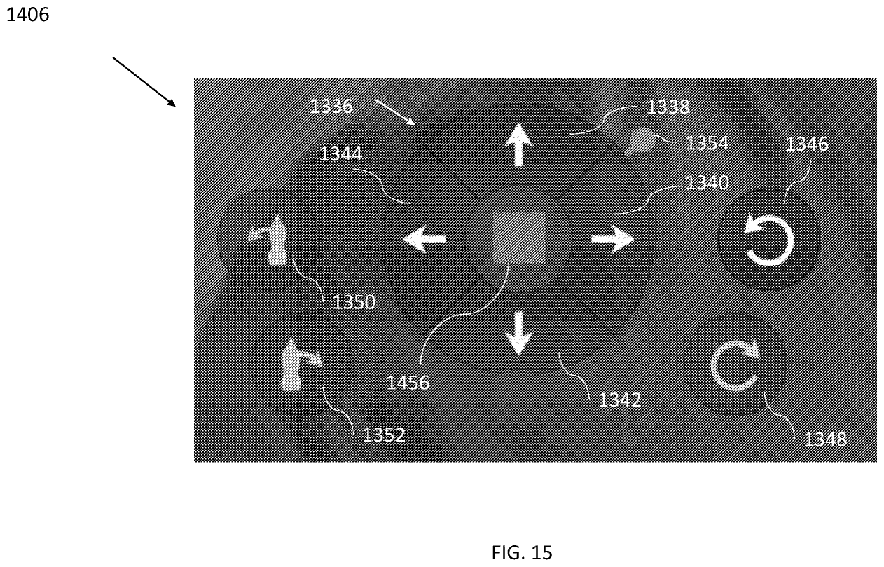

FIG. 15 illustrates another example instruction interface 1406, in accordance with certain embodiments described herein. The instruction interface 1406 is the same as the instruction interface 1306, except that the instruction interface 1406 includes the stop option 1456. The instruction interface 1406 may be displayed after selection of an option from the instruction interface 1306. As will be described below, in response to receiving selection of an option from an instruction interface such as the instruction interface 1306, both the operator GUI 200 and the instructor GUI 300 may display a directional indicator. In some embodiments, in response to receiving a selection of the stop option 1456 from the instruction interface 1406, the instructor GUI 300 may stop displaying the directional indicator. Additionally, in some embodiments, the instructor processing device 122 may issue a command to the operator processing device 104 to stop displaying the directional indicator on the operator GUI 200.

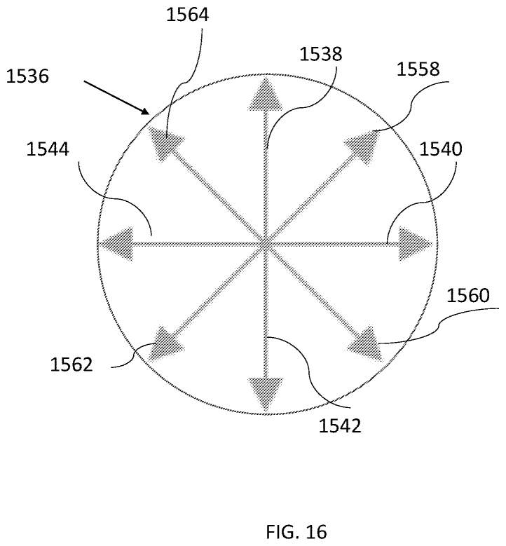

FIG. 16 illustrates another example translation interface 1536, in accordance with certain embodiments described herein. The translation interface 1536 includes an up instruction option 1538, an up-right instruction option 1558, a right instruction option 1540, a down-right instruction option 1560, a down instruction option 1542, a down-left instruction option 1562, a left instruction option 1544, and an up-left instruction option 1564. The orientation indicator 1354 may also be displayed in the same manner as in FIG. 14. In some embodiments, in response to receiving a selection of the right option 1340, the up option 1338, the up-right instruction option 1558, the up instruction option 1538, the up-left instruction option 1564, the left instruction option 1544, the down-left instruction option 1562, the down instruction option 1542, or the down-right instruction option 1560, the instructor processing device 122 may output to the operator processing device 104 an angle for translation corresponding to the selected option (e.g., 0, 45, 90, 135, 180, 225, 270, or 315 degrees, respectively).

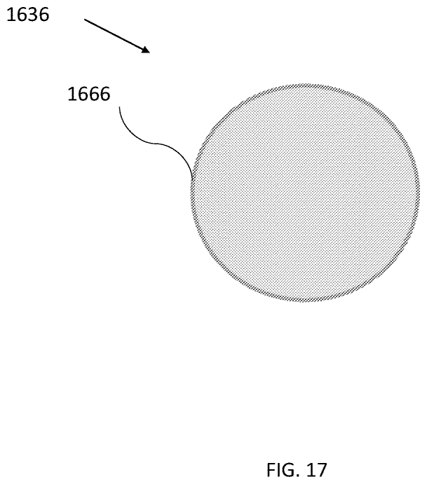

FIG. 17 illustrates another example translation interface 1636, in accordance with certain embodiments described herein. The translation interface 1636 includes a circle 1666. The orientation indicator 1354 may also be displayed in the same manner as in FIG. 14.

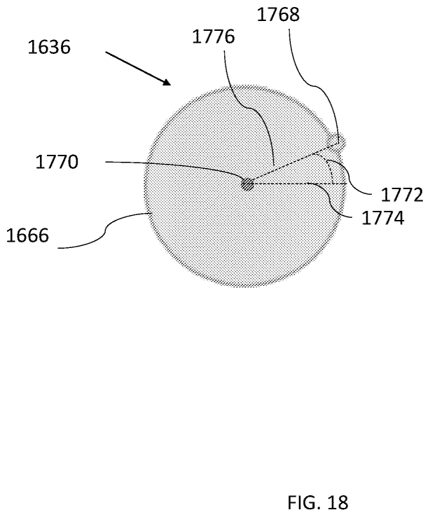

FIG. 18 illustrates an example of operation of the translation interface 1636, in accordance with certain embodiments described herein. In FIG. 18, the operator has selected (e.g., by clicking or touching) the location 1768 along the circumference of the circle 1666. In some embodiments, the location 1768 may be displayed by a marker, while in other embodiments, a marker may not be displayed. The center 1770 of the circle 1666 is also highlighted in FIG. 18 (but may not be actually displayed). In response to receiving the selection by the operator of the location along the circumference of the circle 1666, the instructor processing device 122 may output to the operator processing device 104 an angle for translation corresponding to the angle 1772 between the horizontal rightward-extending radius 1774 of the circle 1666 and a line 1776 extending from the center 1770 of the circle 1666 to the selected location 1768 along the circumference of the circle 1666. (The radius 1774 and the line 1776 may not be displayed.)

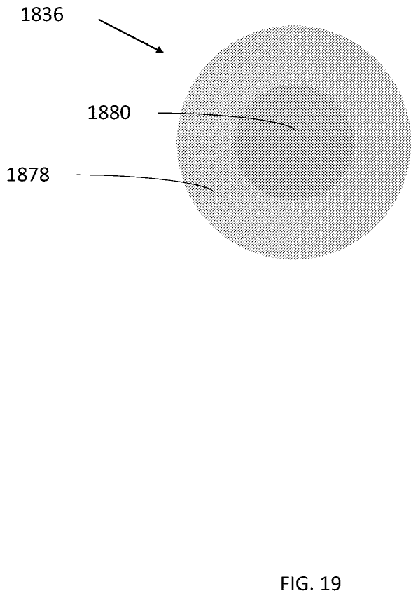

FIG. 19 illustrates another example translation interface 1836, in accordance with certain embodiments described herein. The translation interface 1836 includes an outer circle 1878 and an inner circle 1880. An operator may drag (e.g., by clicking and holding down a button on a mouse while dragging the mouse or by touching and dragging his/her finger or a stylus on a touch-sensitive display screen) the inner circle 1880 within the outer circle 1878.

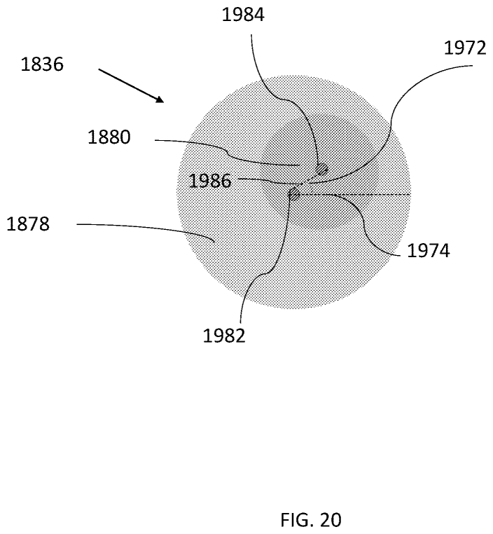

FIG. 20 illustrates an example of operation of the translation interface 1836, in accordance with certain embodiments described herein. In FIG. 20, the operator has dragged the inner circle 1880 to a particular location within the outer circle 1878. The center 1982 of the outer circle 1878 and the center 1984 of the inner circle 1880 are highlighted (but may not actually be displayed). In response to receiving a selection by the operator of the particular location for the inner circle 1880 within the outer circle 1878, the instructor processing device 122 may output to the operator processing device 104 an angle for translation corresponding to the angle 1972 between the horizontal rightward-extending radius 1974 of the outer circle 1878 and a line 1986 extending from the center 1982 of the outer circle 1878 to the center 1984 of the inner circle 1880.

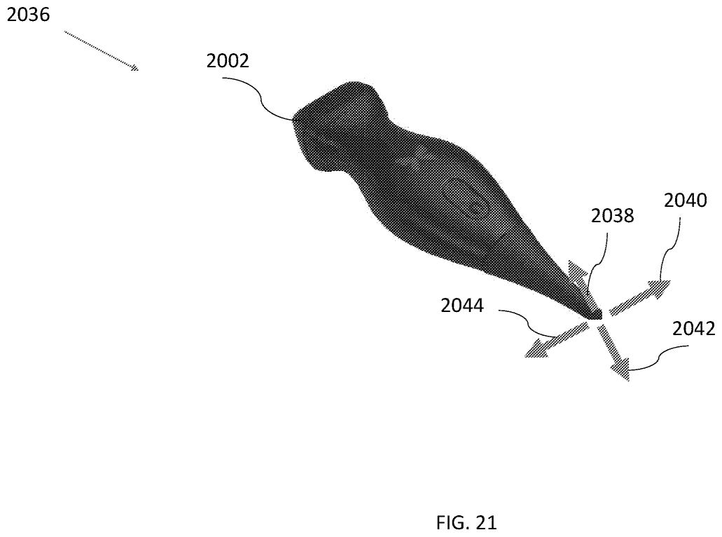

FIG. 21 illustrates another example translation interface 2036, in accordance with certain embodiments described herein. The translation interface 2036 includes an image 2002 of the ultrasound device 102, an up option 2038, a right option 2040, a down option 2042, and a left option 2044. In some embodiments, in response to receiving a selection of the right option 2040, the up option 2038, the left option 2044, or the down option 2042, the instructor processing device 122 may output to the operator processing device 104 an angle for translation corresponding to the selected option (e.g., 0, 90, 180, or 270 degrees, respectively). In some embodiments, the image of the ultrasound device 102 may display the ultrasound device 102 in a fixed orientation. In some embodiments, the image of the ultrasound device 102 may update the orientation of the ultrasound device 102 in the image to match the orientation of the actual ultrasound device 102 relative to the operator processing device 104 (which may be determined as described below).

In some embodiments, in addition to displaying instruction options corresponding to up, down, right, and left, the translation interface 2036 may also display instruction options corresponding to up-right, down-right, down-left, and up-left. In some embodiments, the translation interface 2036 may also display instruction options corresponding to rotations and tilts. In some embodiments, the instructor may select a location around the image of the ultrasound device 102, and the instructor processing device 122 may issue an instruction equivalent to an angle formed by the selected location relative to the right option 2040 (or whichever zero angle is used). In some embodiments, the instructor may first click or touch the tip of the ultrasound device 102 in the image of the ultrasound device 102, and then drag (e.g., by holding down a button on a mouse while dragging the mouse or by touching and dragging his/her finger on a touch-sensitive display screen) to a selected location. The instructor processing device 122 may issue an instruction equivalent to an angle formed by the selected location relative to the right option 2040 (or whichever zero angle is used.

### Pose Determination

The position of the ultrasound device 102 relative to the operator processing device 104 may include components along three degrees of freedom, namely the position of the ultrasound device 102 along the horizontal, vertical, and depth dimensions relative to the operator processing device 104. In some embodiments, determining the horizontal and vertical components of the position of the ultrasound device 102 relative to the operator processing device 104 may constitute determining, for a given frame of video, the horizontal and vertical coordinates of a pixel in the video frame that corresponds to the position of a particular portion of the ultrasound device 102 in the video frame. In some embodiments, the particular portion of the ultrasound device 102 may be the tail of the ultrasound device 102.

In some embodiments, the operator processing device 104 may use a statistical model trained to determine the horizontal and vertical components of the position of the ultrasound device 102 relative to the operator processing device 104. In some embodiments, the statistical model may be trained as a keypoint localization model with training input and output data. Multiple images of the ultrasound device 102 may be inputted to the statistical model as training input data. As training output data, an array of values that is the same size as the inputted image may be inputted to the statistical model, where the pixel corresponding to the location of the tip of the ultrasound device 102 (namely, the end of the ultrasound device 102 opposite the sensor portion) in the image is manually set to a value of 1 and every other pixel has a value of 0. (While values of 1 and 0 are described, other values may be used instead.) Based on this training data, the statistical model may learn to output, based on an inputted image (e.g., a frame of the video of the ultrasound device 102 captured by the operator processing device 104), an array of values that is the same size as the inputted image, where each pixel in the array consists of a probability that that pixel is where the tip of the ultrasound image is located in the inputted image. The operator processing device 104 may then predict that the pixel having the highest probability represents the location of the tip of the ultrasound image and output the horizontal and vertical coordinates of this pixel.

In some embodiments, the statistical model may be trained to use regression to determine the horizontal and vertical components of the position of the ultrasound device 102 relative to the operator processing device 104. Multiple images of the ultrasound device 102 may be inputted to the statistical model as training input data. As training output data, each input image may be manually labeled with two numbers, namely the horizontal and vertical pixel coordinates of the tip of the ultrasound device 102 in the image. Based on this training data, the statistical model may learn to output, based on an inputted image (e.g., a frame of the video of the ultrasound device 102 captured by the operator processing device 104), the horizontal and vertical pixel coordinates of the tip of the ultrasound device 102 in the image.

In some embodiments, the statistical model may be trained as a segmentation model to determine the horizontal and vertical components of the position of the ultrasound device 102 relative to the operator processing device 104. Multiple images of the ultrasound device 102 may be inputted to the statistical model as training input data. As training output data, a segmentation mask may be inputted to the statistical model, where the segmentation mask is an array of values equal in size to the image, and pixels corresponding to locations within the ultrasound device 102 in the image are manually set to 1 and other pixels are set to 0. (While values of 1 and 0 are described, other values may be used instead.) Based on this training data, the statistical model may learn to output, based on an inputted image (e.g., a frame of the video of the ultrasound device 102 captured by the operator processing device 104), a segmentation mask where each pixel has a value representing the probability that the pixel corresponds to a location within the ultrasound device 102 in the image (values closer to 1) or outside the ultrasound device 102 (values closer to 0). Horizontal and vertical pixel coordinates representing a single location of the ultrasound device 102 in the image may then be derived (e.g., using averaging or some other method for deriving a single value from multiple values) from this segmentation mask.

In some embodiments, determining the position of ultrasound device 102 along the depth dimension relative to the operator processing device 104 may include determining the distance of a particular portion (e.g., the tip) of the ultrasound device 102 from the operator processing device 104. In some embodiments, the operator processing device 104 may use a statistical model (which may be the same as or different than any of the statistical models described herein) trained to determine the position of ultrasound device 102 along the depth dimension relative to the operator processing device 104. In some embodiments, the statistical model may be trained to use regression to determine the position of ultrasound device 102 along the depth dimension relative to the operator processing device 104. Multiple images of the ultrasound device 102 may be inputted to the statistical model as training input data. As training output data, each input image may be manually labeled with one number, namely the distance of the tip of the ultrasound device 102 from the operator processing device 104 when the image was captured. In some embodiments, a depth camera may be used to generate the training output data. For example, the depth camera may use disparity maps or structure light cameras. Such cameras may be considered stereo cameras in that they may use two cameras at different locations on the operator processing device 104 that simultaneously capture two images, and the disparity between the two images may be used to determine the depth of the tip of the ultrasound device 102 depicted in both images. In some embodiments, the depth camera may be a time-of-flight camera may be used to determine the depth of the tip of the ultrasound device 102. In some embodiments, the depth camera may generate absolute depth values for the entire video frame, and because the position of the tip of the ultrasound probe in the video frame may be determined using the method described above, the distance of the tip of the ultrasound probe from the operator processing device 104 may be determined. Based on this training data, the statistical model may learn to output, based on an inputted image (e.g., a frame of the video of the ultrasound device 102 captured by the operator processing device 104), the distance of the tip of the ultrasound device 102 from the operator processing device 104 when the image was captured. In some embodiments, the operator processing device 104 may use a depth camera to directly determine the depth of the tip of the ultrasound device 102, in the same manner discussed above for generating training data, without using a statistical model specifically trained to determine depth. In some embodiments, the operator processing device 104 may assume a predefined depth as the depth of the tip of the ultrasound device 102 relative to the operator processing device 104.

In some embodiments, using camera intrinsics (e.g., focal lengths, skew coefficient, and principal points), the operator processing device 104 may convert the horizontal and vertical pixel coordinates of the tip of the ultrasound device 102 into the horizontal (x-direction) and vertical (y-direction) distance of the tip of the ultrasound device 102 relative to the operator processing device 104 (more precisely, relative to the camera of the operator processing device 104). In some embodiments, the operator processing device 104 may use the distance of the tip of the ultrasound device 102 from the operator processing device 104 (determined using any of the methods above) to convert the horizontal and vertical pixel coordinates of the tip of the ultrasound device 102 into the horizontal (x-direction) and vertical (y-direction) distance of the tip of the ultrasound device 102 relative to the operator processing device 104. It should be appreciated that while the above description has focused on using the tip of the ultrasound device 102 to determine the position of the ultrasound device 102, any feature on the ultrasound device 102 may be used instead.

In some embodiments, an auxiliary marker on the ultrasound device 102 may be used to determine the distances of that feature relative to the operator processing device 104 in the horizontal, vertical, and depth-directions based on the video of the ultrasound device 102 captured by the operator processing device 104, using pose estimation techniques and without using statistical models. For example, the auxiliary marker may be a marker conforming to the ArUco library, a color band, or some feature that is part of the ultrasound device 102 itself.

The orientation of the ultrasound device 102 relative to the operator processing device 104 may include three degrees of freedom, namely the roll, pitch, and yaw angles relative to the operator processing device 104. In some embodiments, the operator processing device 104 may use a statistical model (which may be the same as or different than any of the statistical models described herein) trained to determine the orientation of the ultrasound device 102 relative to the operator processing device 104. In some embodiments, the statistical model may be trained to use regression to determine the orientation of the ultrasound device 102 relative to the operator processing device 104. Multiple images of the ultrasound device 102 may be inputted to the statistical model as training input data. As training output data, each input image may be manually labeled with three numbers, namely the roll, pitch, and yaw angles of the ultrasound device 102 relative to the operator processing device 104 when the image was captured. In some embodiments, the training output data may be generated using sensor data from the ultrasound device 102 and sensor data from the operator processing device 104. The sensor data from the ultrasound device 102 may be collected by a sensor on the ultrasound device 102 (e.g., the sensor 106). The sensor data from the operator processing device 104 may be collected by a sensor on the operator processing device 104 (e.g., the sensor 118). The sensor data from each device may describe the acceleration of the device (e.g., as measured by an accelerometer), the angular velocity of the device (e.g., as measured by a gyroscope), and/or the magnetic field in the vicinity of the device (e.g., as measured by a magnetometer). Using sensor fusion techniques (e.g., based on Kalman filters, complimentary filters, and/or algorithms such as the Madgwick algorithm), this data may be used to generate the roll, pitch, and yaw angles of the device relative to a coordinate system defined by the directions of the local gravitational acceleration and the local magnetic field. If the roll, pitch, and yaw angles of each device are described by a rotation matrix, then multiplying the rotation matrix of the operator processing device 104 by the inverse of the rotation matrix of the ultrasound device 102 may produce a matrix describing the orientation (namely, the roll, pitch, and yaw angles) of the ultrasound device 102 relative to the operator processing device 104. Based on this training data, the statistical model may learn to output, based on an inputted image (e.g., a frame of the video of the ultrasound device 102 captured by the operator processing device 104), the orientation of the ultrasound device 102 relative to the operator processing device 104 when the image was captured. This method will be referred to below as the “statistical model method.”

In some embodiments, the operator processing device 104 may use, at any given time, the sensor data from the ultrasound device 102 and the sensor data from the processing to directly determine orientation at that particular time, without using a statistical model. In other words, at a given time, the operator processing device 104 may use the sensor data collected by the ultrasound device 102 at that time and the sensor data collected by the operator processing device 104 at that time to determine the orientation of the ultrasound device 102 relative to the operator processing device 104 at that time (e.g., using sensor fusion techniques as described above). This method will be referred to below as the “sensor method.”

In some embodiments, if the operator processing device 104 performs the sensor method using data from accelerometers and gyroscopes, but not magnetometers, on the ultrasound device 102 and the operator processing device 104, the operator processing device 104 may accurately determine orientations of the ultrasound device 102 and the operator processing device 104 except for the angle of the devices around the direction of gravity. It may be helpful not to use magnetometers, as this may obviate the need for sensor calibration, and because external magnetic fields may interfere with measurements of magnetometers on the ultrasound and operator processing device 104. In some embodiments, if the operator processing device 104 performs the statistical model method, the operator processing device 104 may accurately determine the orientation of the ultrasound device 102 relative to the operator processing device 104, except that the statistical model method may not accurately detect when the ultrasound device 102 rotates around its long axis as seen from the reference frame of the operator processing device 104. This may be due to symmetry of the ultrasound device 102 about its long axis. In some embodiments, the operator processing device 104 may perform both the statistical model method and the sensor method, and combine the determinations from both methods to compensate for weaknesses of either method. For example, as described above, using the sensor method, the operator processing device 104 may not accurately determine orientations of the ultrasound device 102 and the operator processing device 104 around the direction of gravity when not using magnetometers. Since, ultimately, determining the orientation of the ultrasound device 102 relative to the operator processing device 104 may be desired, it may only be necessary to determine the orientation of the ultrasound device 102 around the direction of gravity as seen from the reference frame of the operator processing device 104. Thus, in some embodiments, the operator processing device 104 may use the sensor method (using just accelerometers and gyroscopes) for determining orientation of the ultrasound device 102 relative to the operator processing device 104 except for determining the orientation of the ultrasound device 102 around the direction of gravity as seen from the reference frame of the operator processing device 104, which the operator processing device 104 may use the statistical model to determine. In such embodiments, rather than using a statistical model trained to determine the full orientation of the ultrasound device 102 relative to the operator processing device 104, the statistical model may be specifically trained to determine, based on an inputted image, the orientation of the ultrasound device 102 around the direction of gravity as seen from the reference frame of the operator processing device 104. In general, the operator processing device 104 may combine determinations from the statistical model method and the sensor method to produce a more accurate determination.

In some embodiments, a statistical model may be trained to locate three different features of the ultrasound device 102 in the video of the ultrasound device 102 captured by the operator processing device 104 (e.g., using methods described above for locating a portion of an ultrasound device 102, such as the tip, in an image), from which the orientation of the ultrasound device 102 may be uniquely determined.

In some embodiments, the training output data for both position and orientation may be generated by manually labeling, in images of ultrasound devices captured by operator processing devices (the training input data), key points on the ultrasound device 102, and then an algorithm such as Solve PnP may determine, based on the key points, the position and orientation of the ultrasound device 102 relative to the operator processing device 104. A statistical model may be trained on this training data to output, based on an inputted image of an ultrasound device 102 captured by an operator processing device, the position and orientation of the ultrasound device 102 relative to the operator processing device 104.

It should be appreciated that determining a position and/or orientation of the ultrasound device 102 relative to the operator processing device 104 may include determining any component of position and any component of orientation. For example, it may include determining only one or two of the horizontal, vertical, and depth dimensions of position and/or only one or two of the roll, pitch, and yaw angles.

### Displaying Instructions

The above description has described how particular instructions can be selected by an instructor from instruction interfaces. As described, the instructor processing device 122 may output to the operator processing device 104 rotation instructions, tilt instructions, and translation instructions. In some embodiments, a rotation instruction may either be an instruction to perform a clockwise rotation or a counterclockwise rotation instruction. In some embodiments, a tilt instruction may either be an instruction to tilt the face 688 of the ultrasound device 102 towards the subject 208 or to tilt the face 690 of the ultrasound device 102 towards the subject 208. In some embodiments, a translation instruction may include an instruction to translate the ultrasound device 102 in a direction corresponding to a particular angle.

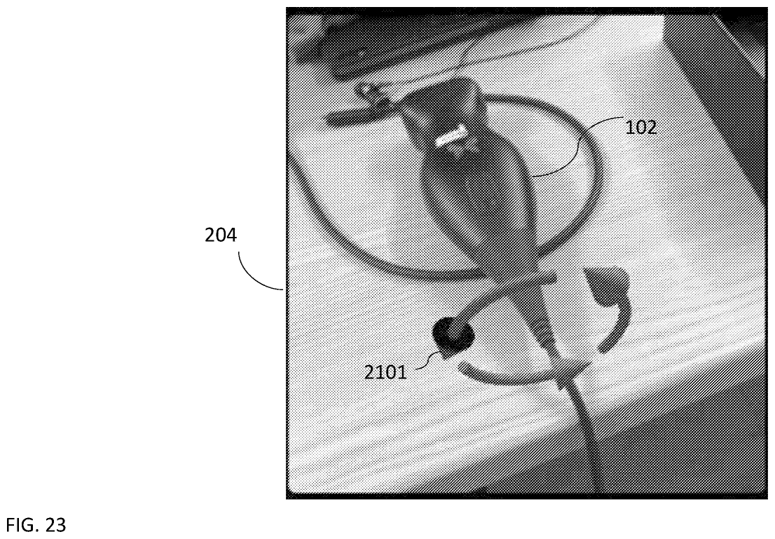

In some embodiments, upon selection of an instruction from an instruction interface, the instructor processing device 122 may display a directional indicator in the operator video 204 on the instructor GUI (e.g., the instructor GUI 300) corresponding to that instruction. Additionally, the instructor processing device 122 may transmit the instruction to the operator processing device 104 which may then the display a directional indicator in the operator video 204 on the operator GUI (e.g., the operator GUI 200) corresponding to that instruction. The combination of the directional indicator and the operator video 204 (and, as will be discussed below, an orientation indicator such as an orientation ring in some embodiments) may be considered an augmented reality display. The directional indicator may be displayed in the operator video 204 such that the directional indicator appears to be a part of the real-world environment in the operator video 204. When displaying directional indicators corresponding to a particular instruction, the instructor processing device 122 and the operator processing device 104 may display one or more arrows that are positioned and oriented in the operator video 204 based on the pose determination described above. In some embodiments, the instructor processing device 122 may receive, from the operator processing device 104, the pose of the ultrasound device 102 relative to the operator processing device 104. Further description of displaying directional indicators may be found with reference to FIGS. 23-25.

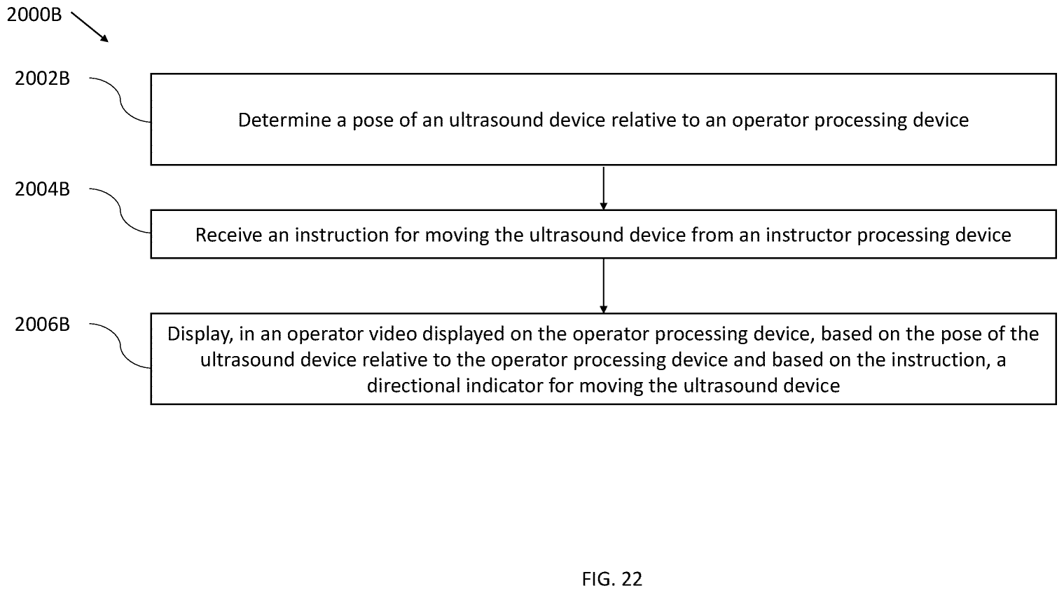

FIG. 22 illustrates an example process 2000B for displaying instructions for moving an ultrasound device 102 on the operator processing device 104, in accordance with certain embodiments described herein. The process 2000B may be performed by the operator processing device 104.

In act 2002B, the operator processing device 104 determines a pose of the ultrasound device 102 relative to the operator processing device 104. The operator processing device 104 may use, for example, any of the methods for determining pose described above. The process 2000B proceeds from act 2002B to act 2004B.

In act 2004B, the operator processing device 104 receives an instruction for moving the ultrasound device 102 from the instructor processing device 122. As described above, an instructor may select an instructor for moving the ultrasound device 102 from an instruction interface, and the instructor processing device 122 may transmit the instruction to the operator processing device 104. The process 2000B proceeds from act 2002B to act 2004B.

In act 2006B, the operator processing device 104 displays, in the operator video 204 displayed on the operator processing device 104, based on the pose of the ultrasound device 102 relative to the operator processing device 104 (determined in act 2002B) and based on the instruction (received in act 2004B), a directional indicator for moving the ultrasound device 102. Further description of displaying directional indicators may be found below. The combination of the operator video 204 and the directional indicator may constitute an augmented reality display.

FIG. 23 illustrates an example of the operator video 204, in accordance with certain embodiments described herein. The operator video 204 may be displayed in the operator GUI 200. The operator video 204 in FIG. 23 displays the ultrasound device 102 and a directional indicator 2101. The directional indicator 2101 includes multiple arrows pointing in a counterclockwise direction, corresponding to an instruction to rotate the ultrasound device 102 counterclockwise. The directional indicator 2101 is centered approximately at the tail of the ultrasound device 102 and oriented approximately within a plane orthogonal to the longitudinal axis of the ultrasound device 102. To display the directional indicator 2101 in this way, a default position and orientation of the directional indicator 2101 in three-dimensional space may be known for a particular default pose of the ultrasound device 102 relative to the operator processing device 104, such that the directional indicator 2101 is centered approximately at the tail of the ultrasound device 102 and oriented approximately within a plane orthogonal to the longitudinal axis of the ultrasound device 102. Then, the operator processing device 104 may translate, rotate, and/or tilt the directional indicator 2101 in three-dimensional space from the default position and orientation based on the difference between the current pose (as determined using the methods described above) and the default pose of the ultrasound device 102 relative to the operator processing device 104, and then project the three-dimensional position and orientation of the directional indicator 2101 into two-dimensional space for display in the operator video 204.

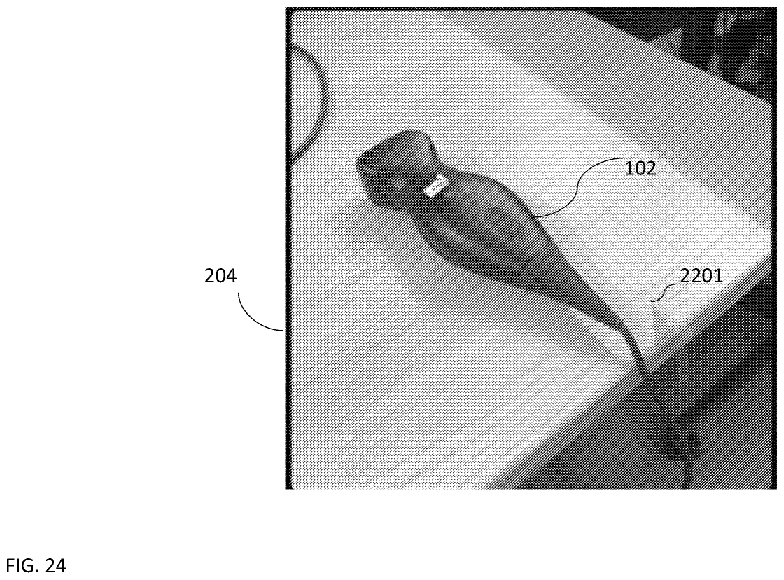

FIG. 24 illustrates an example of the operator video 204, in accordance with certain embodiments described herein. The operator video 204 may be displayed in the operator GUI 200. The operator video 204 in FIG. 24 displays the ultrasound device 102 and a directional indicator 2201. The directional indicator 2201 includes an arrow indicating a tilt of the face 688 of the ultrasound device 102, corresponding to an instruction to tilt the face 688 of the ultrasound device 102. The directional indicator 2201 is located approximately at the tail of the ultrasound device 102 and oriented to point approximately along the face 688 of the ultrasound device 102 within a plane parallel to the longitudinal axis of the ultrasound device 102. To display the directional indicator 2201 in this way, a default position and orientation of the directional indicator 2201 in three-dimensional space may be known for a particular default pose of the ultrasound device 102 relative to operator processing device 104, such that the directional indicator 2201 is located approximately at the tail of the ultrasound device 102 and oriented such that the directional indicator 2201 points approximately along the face 688 of the ultrasound device 102 within a plane parallel to the longitudinal axis of the ultrasound device 102. Then, the operator processing device 104 may translate, rotate, and/or tilt the directional indicator 2201 in three-dimensional space from the default position and orientation based on the difference between the current pose (as determined using the methods described above) and the default pose the default pose of the ultrasound device 102 relative to the operator processing device 104, and then project the three-dimensional position and orientation of the directional indicator 2201 into two-dimensional space for display in the operator video 204.

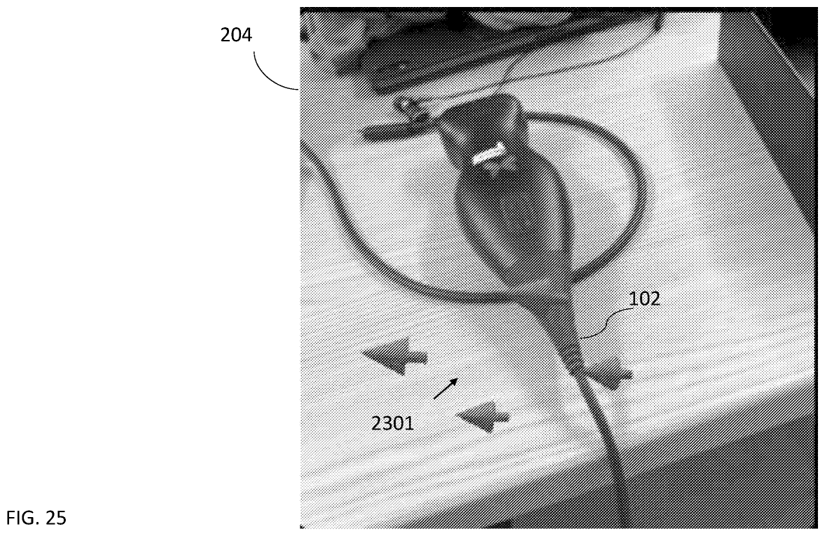

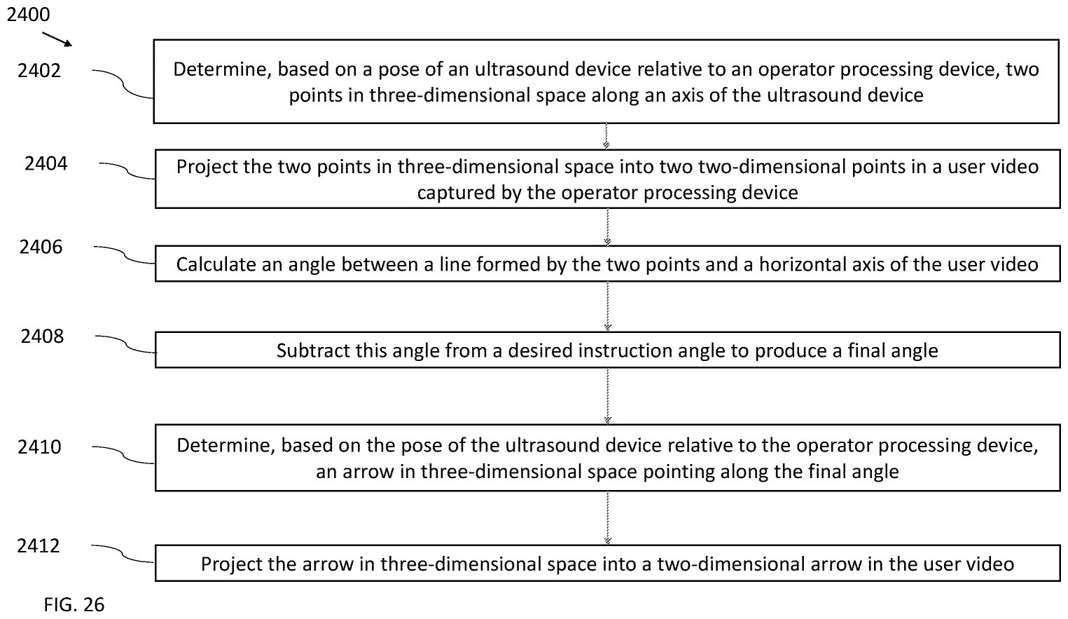

FIG. 25 illustrates an example of the operator video 204, in accordance with certain embodiments described herein. The operator video 204 may be displayed in the operator GUI 200. The operator video 204 in FIG. 25 displays the ultrasound device 102 and a directional indicator 2301. The directional indicator 2301 includes multiple arrows pointing in a particular direction, corresponding to an instruction to translate the ultrasound device 102 in that direction. FIG. 26 describes an example of how to display the directional indicator 2301 in more detail.

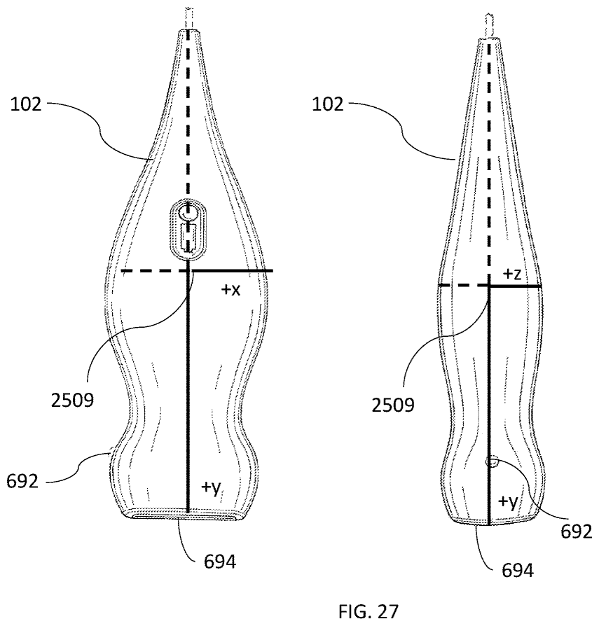

FIG. 26 illustrates an example process 2400 for displaying a directional indicator for translating the ultrasound device 102, in accordance with certain embodiments described herein. The process 2400 may be performed by either the operator processing device 104 or the instructor processing device 122. For simplicity, the below description will describe the process 2400 as being performed by a processing device. FIG. 27 illustrates an example coordinate system for the ultrasound device 102, in accordance with certain embodiments described herein. FIG. 27 illustrates an x-axis, y-axis, and z-axis of the coordinate system, the positive direction of each axis, and an origin 2509 of the ultrasound device 102. Referring back to FIG. 26, all three-dimensional coordinates are given with the x-coordinate first, the y-coordinate second, and optionally the z-coordinate third (where x-, y-, and z-coordinates refer to position along the x-, y-, and z-axes, respectively, of the ultrasound device 102 in FIG. 27 relative to the origin 2509).

In act 2402, the processing device determines, based on a pose of the ultrasound device 102 to the operator processing device 104, two points in three-dimensional space along an axis of the ultrasound device 102. The pose of the ultrasound device 102 relative to the operator processing device 104 may have been determined using the methods described above. In some embodiments, the operator processing device 104 may determine a point P1 at (0, 0, 0), where point P1 is at a center of the ultrasound device 102, and a point P2 at (x, 0, 0), where x is any positive offset (e.g., 1) along the x-axis of the ultrasound device 102 and where, as illustrated in FIG. 27, the positive x-axis of the ultrasound device 102 is parallel to a line from the longitudinal axis of the ultrasound device 102 to the marker 692. The process 2400 proceeds from act 2402 to act 2404.

In act 2404, the processing device project the two points in three-dimensional space into two two-dimensional points in the operator video 204 captured by the operator processing device 104. In some embodiments, the processing device may rotate P2 by the three-dimensional rotation of the ultrasound device 102 relative to the operator processing device 104 (as determined using the methods described above for determining pose) with P1 being the origin of rotation. In some embodiments, the processing device may apply a rotation matrix to P2, where the rotation matrix describes the rotation of the ultrasound device 102 relative to the operator processing device 104. In some embodiments, the processing device may use camera intrinsics (e.g., focal lengths, skew coefficient, and principal points) to perform the projection. Let the coordinates of the projection of P1 be P1′ at (P1′ₓ, P1′_{y}) and the coordinates of the projection of P2 be P2′ at (P2′ₓ, P2′_{y}), where the first coordinate is along the horizontal axis of the operator video 204 and the second coordinate is along the vertical axis of the operator video 204. The process 2400 proceeds from act 2404 to act 2406.

In act 2406, the processing device calculates an angle between a line formed by the two points and an axis (e.g., the horizontal axis, although other axes may be used instead) of the operator video 204. In some embodiments, the processing device may determine a circle with center P1′ and with P2′ along the circumference of the circle. In other words, the distance between P1′ and P2′ is the radius of a circle. The processing device may determine a point P3 at (P1′ₓ+radius of the circle, P1′_{y}). In other words, P3 is on the circumference of the circle, directly offset to the right from P1′ in the operator video 204. The processing device may then calculate the angle between P1′-P3′ (i.e., a line extending between P1′ and P3′) and P1′-P2′ (i.e., a line extending between P1′ and P2′). The process 2400 proceeds from act 2406 to act 2408.

In act 2408, the processing device subtracts this angle (i.e., the angle calculated in act 2406) from a desired instruction angle to produce a final angle. The selected instruction angle may be the angle selected from any of the translation interfaces described herein. For example, as described with reference to the translation interface 1006, in some embodiments, in response to cessation of a dragging movement (e.g., releasing a finger or releasing a mouse button), the cursor 1032 and the arrow 1026 may cease to move, and the translation interface 1006 may display the arrow 1026 in a different color. This may correspond to selection of the angle of the arrow 1026 with respect to the horizontal axis of the circle 1034 (although other axes may be used instead). The final angle resulting from the subtraction of the angle calculated in act 2416 from the selected instruction angle may be referred to as A. The process 2400 proceeds from act 2408 to act 2410.

In act 2410, the processing device determines, based on the pose of the ultrasound device relative to the operator processing device, an arrow in three-dimensional space pointing along the final angle. In some embodiments, the processing device may determine an arrow to begin at (0,0,0), namely the origin of the ultrasound device 102, and end at (L cos A, 0, L sin A), where L is the length of the arrow and A is the final angle calculated in act 2408. The process 2400 proceeds from act 2410 to act 2412.

In act 2412, the processing device projects the arrow in three-dimensional space (determined in act 2410) into a two-dimensional arrow in the operator video 204. In some embodiments, the processing device may rotate the arrow by the rotation matrix that describes the orientation of the ultrasound device 102 relative to the operator processing device 104 and project the three-dimensional arrow into a two-dimensional arrow in the operator video 204 (e.g., using camera intrinsics, as described above with reference to act 2404).

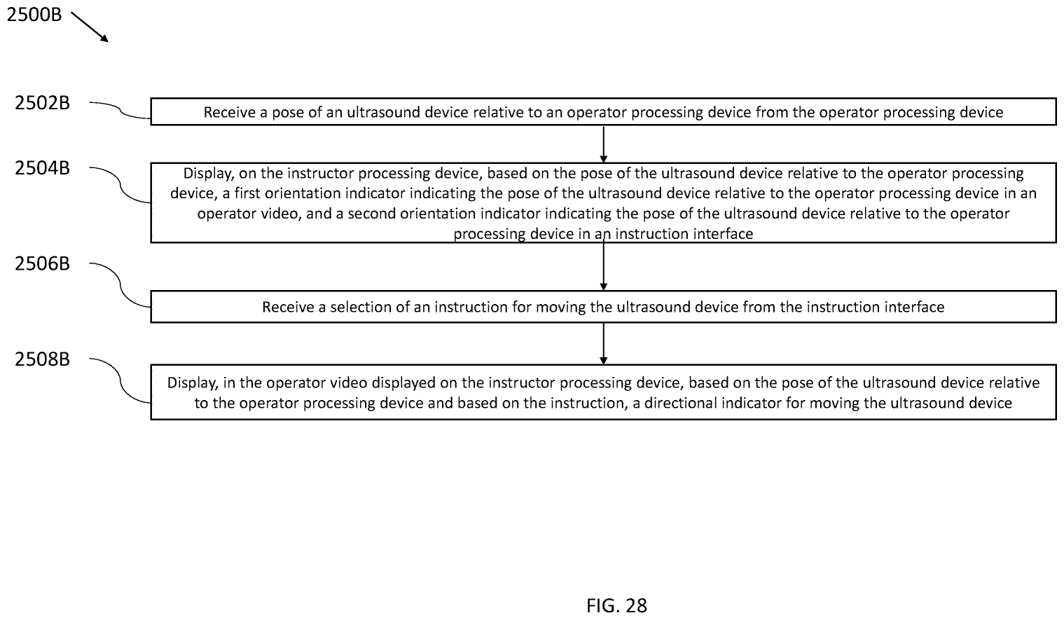

FIG. 28 illustrates an example process 2500B for displaying instructions for moving the ultrasound device 102 on the instructor processing device 122, in accordance with certain embodiments described herein. The process 2500B may be performed by the instructor processing device 124.

In act 2502B, the instructor processing device 122 receives, from the operator processing device 104, a pose of the ultrasound device 102 relative to the operator processing device 104. The operator processing device 104 may use, for example, any of the methods for determining pose described above, and transmit the pose to the instructor processing device 122. The process 2500B proceeds from act 2502B to act 2504B.

In act 2504B, the instructor processing device 122 displays, based on the pose of the ultrasound device 102 relative to the operator processing device 104 (received in act 2502B), a first orientation indicator indicating the pose of the ultrasound device 102 relative to the operator processing device 104, where the first orientation indicator is displayed in the operator video 204 on the instructor processing device. The first orientation indicator may be, for example, the orientation ring 2607 described below. The instructor processing device 122 also displays, based on the pose of the ultrasound device 102 relative to the operator processing device 104 (received in act 2502B), a second orientation indicator indicating the pose of the ultrasound device 102 relative to the operator processing device 104, where the second orientation indicator is displayed in an instruction interface on the instructor processing device 122. The second orientation indicator may be, for example, the orientation indicator 524 or 1354, and the instruction interface may be any of the instruction interfaces described herein. Further description of displaying the first orientation indicator and the second orientation indicator may be found below. The process 2500B proceeds from act 2504B to act 2506B.

In act 2506B, the instructor processing device 122 receives a selection of an instruction for moving the ultrasound device 102 from the instruction interface. Further description of receiving instructions may be found with reference to any of the instruction interfaces described herein. The process 2500B proceeds from act 2506B to act 2508B.

In act 2508B, the instructor processing device 122 displays, in the operator video 204 displayed on the operator processing device 104, based on the pose of the ultrasound device 102 relative to the operator processing device 104 (received in act 2502B) and based on the instruction (received in act 2006B), a directional indicator for moving the ultrasound device 102. Further description of displaying directional indicators may be found below. The combination of the operator video 204 and the directional indicator may constitute an augmented reality display.

In some embodiments, the instructor processing device 122 may just perform acts 2502B and 2504B. For example, an instruction may not yet have been selected. In some embodiments, the instructor processing device 122 may only display the first orientation indicator, or only display the second orientation indicator, at act 2504B. In some embodiments, the instructor processing device 122 may not display either the first orientation indicator or the second orientation indicator (i.e., act 2504B may be absent).

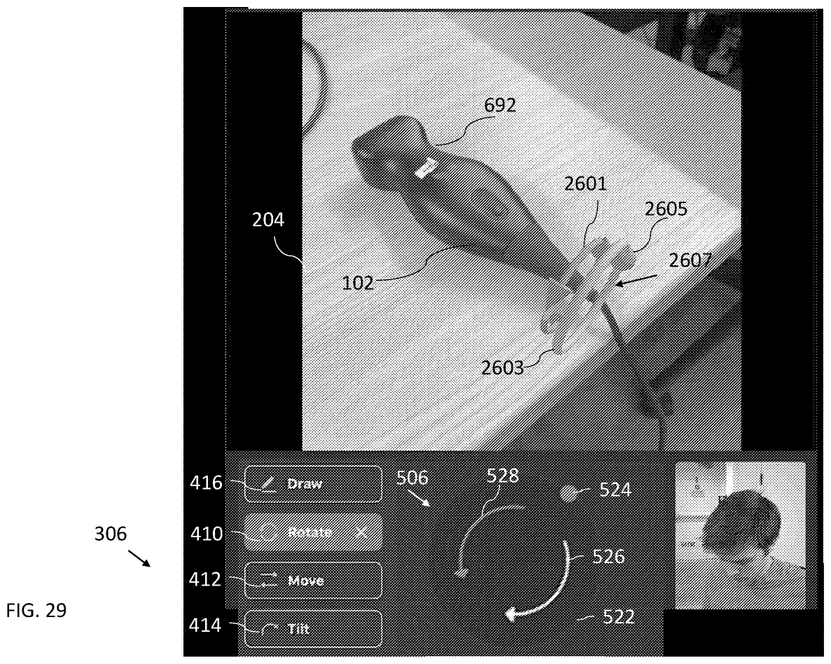

FIG. 29 illustrates an example of the operator video 204 and the instruction interface 306, in accordance with certain embodiments described herein. The operator video 204 and the instruction interface 306 may be displayed in the instructor GUI 300. The operator video 204 may be displayed in the instructor GUI 300. The operator video 204 in FIG. 29 displays the ultrasound device 102, a directional indicator 2601, and an orientation ring 2607. The directional indicator 2601 includes multiple arrows pointing in a counterclockwise direction, corresponding to an instruction to rotate the ultrasound device 102 counterclockwise. The directional indicator 2601 may be displayed in the same manner as the directional indicator 2101.

The orientation ring 2607 is an orientation indicator that includes a ring 2603 and a ball 2605. The orientation ring 2607 may generally indicate in the operator video 204 the pose of the ultrasound device 102 relative to the operator processing device 104 and may particularly highlight the orientation of the marker 692 on the ultrasound device 102 relative to the operator processing device 104. The ring 2603 is centered approximately at the tail of the ultrasound device 102 and oriented approximately within a plane orthogonal to the longitudinal axis of the ultrasound device 102. The ball 2605 may be located on the ring 2603 such that a line from the ring 2603 to the marker 692 on the ultrasound device 102 is parallel to the longitudinal axis of the ultrasound device 102. Further description of displaying the orientation ring 2607 may be found with reference to the process 3000. The form of the orientation ring 2607 is non-limiting and other indicators of the pose of the ultrasound device 102 and/or the pose of the marker 692 relative to the operator processing device 104 may be used.

As can be seen in FIG. 29, the position of the orientation indicator 524 around the circle 522 in the rotation interface 506 and the position of the ball 2605 on the ring 2603 in the operator video 204 corresponds to the pose of the marker 692 of the ultrasound device 102 in the operator video 204 (or in other words, the pose of the marker 692 relative to the camera of the operator processing device 104). (While the marker 692 is not visible in FIG. 29, its position is indicated.) Furthermore, as can be seen in FIG. 29, the selected counterclockwise option 528 in the rotation interface 506 corresponds to the counterclockwise-pointing directional indicator 2601.

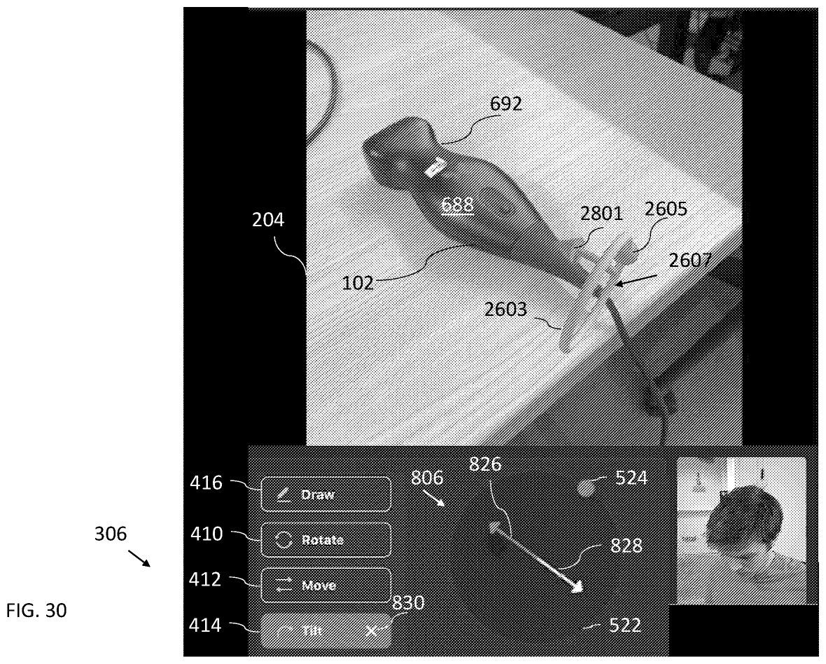

FIG. 30 illustrates an example of the operator video 204 and the instruction interface 306, in accordance with certain embodiments described herein. The operator video 204 and the instruction interface 306 may be displayed in the instructor GUI 300. The operator video 204 in FIG. 30 displays the ultrasound device 102, a directional indicator 2701, and the orientation ring 2607. The directional indicator 2701 includes an arrow indicating a tilt of the face 688 of the ultrasound device 102, corresponding to an instruction to tilt the face 688 of the ultrasound device 102. The directional indicator 2701 may be displayed in the same manner as the directional indicator 2201. As can be seen in FIG. 30, the position of the orientation indicator 524 around the circle 522 in the tilt interface 806 and the position of the ball 2605 on the ring 2603 in the operator video 204 corresponds to the pose of the marker 692 of the ultrasound device 102 in the operator video 204 (or in other words, the pose of the marker 692 relative to the camera of the operator processing device 104). (While the marker 692 is not visible in FIG. 30, its position is indicated.) Furthermore, as can be seen in FIG. 30, the selected tilt option 826 in the tilt interface 806 corresponds to the face 688 of the ultrasound device 102 which the directional indicator 2801 indicates should be tilted.

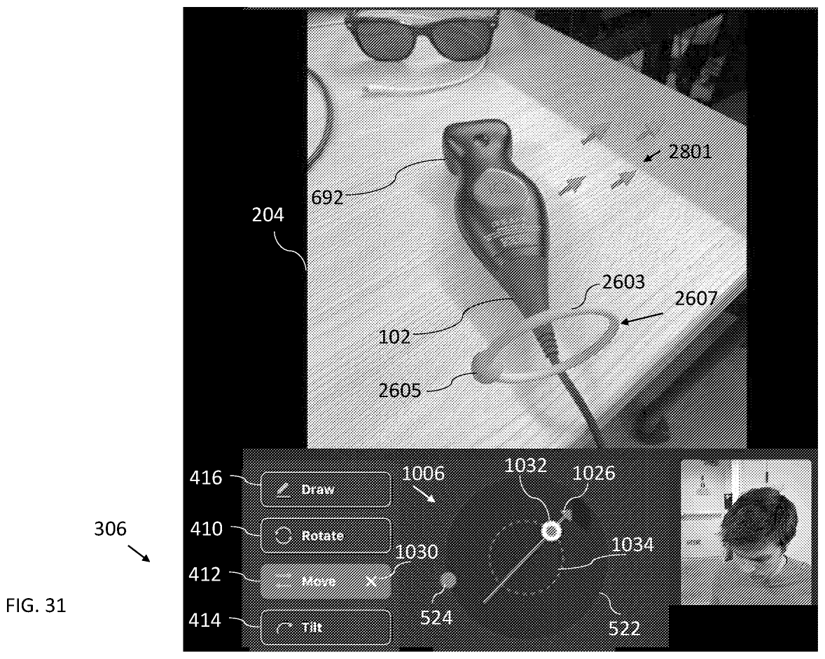

FIG. 31 illustrates an example of the operator video 204 and the instruction interface 306, in accordance with certain embodiments described herein. The operator video 204 and the instruction interface 306 may be displayed in the instructor GUI 300. The operator video 204 in FIG. 31 displays the ultrasound device 102, a directional indicator 2801, and the orientation ring 2607. The directional indicator 2801 includes multiple arrows pointing in a particular direction, corresponding to an instruction to translate the ultrasound device 102 in that direction. The directional indicator 2801 may be displayed in the same manner as the directional indicator 2301. As can be seen in FIG. 31, the position of the orientation indicator 524 around the circle 522 in the translation interface 1006 and the position of the ball 2605 on the ring 2603 in the operator video 204 corresponds to the pose of the marker 692 of the ultrasound device 102 in the operator video 204 (or in other words, the pose of the marker 692 relative to the camera of the operator processing device 104). Furthermore, as can be seen in FIG. 31, the direction of the arrow 1026 in the translation interface 1006 corresponds to the direction of the directional indicator 2801.

In some embodiments, the orientation ring 2607 may not be displayed. In some embodiments, the orientation ring 2607 may be included in the operator video 204 in the operator GUI 200 as well. In some embodiments, while the operator has preliminarily selected an instruction from an instruction interface, but not yet finally selected it, a preview directional indicator may be displayed on the instructor GUI. The preview directional indicator may be the same as a directional indicator displayed based on a final selection, but may differ in some characteristic such as color or transparency. The preview directional indicator may be displayed until the operator changes the preliminary selection or makes a final selection. The instructor processing device 122 may not output an instruction to the operator processing device 104 until the instruction has been finally selected.

For example, in the rotation interface 506, the tilt interface 806, and the translation interfaces 1306, 1406, 1506, and 2036, in some embodiments, touching a finger or stylus to an option but not lifting the finger or stylus up from the option may be a preliminary selection and lifting the finger or stylus up may be a final selection. In some embodiments, holding down a mouse button while pointing a mouse cursor at an option may be a preliminary selection and releasing the mouse button may be a final selection. In the translation interface 1006, in some embodiments touching and dragging the cursor 532 with a finger or stylus, but not releasing the finger or stylus, may be a preliminary selection and lifting the finger or stylus from the cursor 532 may be a final selection. In some embodiments, holding down a mouse button while pointing a mouse cursor at the cursor 532 may be a preliminary selection and releasing the mouse button may be a final selection. In the translation interface 1636, in some embodiments, touching a finger or stylus to a location along the circumference of the circle 1666 but not lifting the finger or stylus up from the option may be a preliminary selection and lifting the finger or stylus up may be a final selection. In some embodiments, holding down a mouse button while pointing a mouse cursor at a location along the circumference of the circle 1666 may be a preliminary selection and releasing the mouse button may be a final selection. In the translation interface 1836, in some embodiments touching and dragging the inner circle 1880 with a finger or stylus, but not releasing the finger or stylus, may be a preliminary selection and lifting the finger or stylus from the inner circle 1880 may be a final selection. In some embodiments, touching and dragging the inner circle 1880 with a finger or stylus may be a preliminary selection and touching a second finger to the inner circle 1880 may be a final selection. In some embodiments, holding down a mouse button while pointing and dragging a mouse cursor may be a preliminary selection and releasing the mouse button may be a final selection. In some embodiments, the length of an arrow generated as a directional indicator based on a selection from the translation interface 1836 may be equivalent to or proportional to the distance from the center 1982 of the outer circle 1878 to the center 1984 of the inner circle 1880. In the translation interface 2036, in embodiments in which the instructor may first click or touch the tip of the ultrasound device 102 in the image of the ultrasound device 102, and then drag a finger or stylus to a selected location, the dragging may be preliminary selection, and lifting the finger or stylus from the inner circle 1880 may be a final selection. In some embodiments, holding down a mouse button while pointing and dragging a mouse cursor may be a preliminary selection and releasing the mouse button may be a final selection. The instructor processing device 122 may issue an instruction equivalent to an angle formed by the selected location relative to the right option 2040 (or whichever zero angle is used. In some embodiments, the length of an arrow generated as a directional indicator based on a selection from the translation interface 2036 may be equivalent to or proportional to the dragging distance.

As described above, in some embodiments, the operator video 204 as displayed in the operator GUI 200 may be flipped horizontally from the operator video 204 as displayed in the instructor GUI 300. When such flipping occurs, when the instructor processing device 122 receives selection of an instruction to move the ultrasound device 102 left (for example) from the perspective of the operator video 204 in the instructor GUI 300, the corresponding directional indicator displayed on the instructor GUI 300 may point to the left in the operator video 204 in the instructor GUI 300, but point to the right in the operator video 204 in the operator GUI 200. Similarly, an instruction to move the ultrasound device 102 right (for example) from the perspective of the operator video 204 in the instructor GUI 300 may point to the right in the operator video 204 in the instructor GUI 300 but point to the left in the operator video 204 in the operator GUI 200 (and similarly for instructions to tilt the ultrasound device 102 left or right). Furthermore, an instruction to rotate the ultrasound device 102 counterclockwise from the perspective of the operator video 204 in the instructor GUI 300 may appear counterclockwise in the operator video 204 in the instructor GUI 300 but clockwise in the operator video 204 in the operator GUI 200, and an instruction to rotate the ultrasound device 102 clockwise from the perspective of the operator video 204 in the instructor GUI 300 may appear clockwise in the operator video 204 in the instructor GUI 300 but counterclockwise in the operator video 204 in the operator GUI 200. Generally, displaying directional indicators may include horizontally flipping the directional indicator. In some embodiments, directional indicators may be animated.

In some embodiments in which directional indicators for translation are displayed based on the orientation of the ultrasound device 102 relative to the operator processing device 104, if a directional indicator for translation is displayed and then the ultrasound device 102 changes its orientation relative to the operator processing device 104, the absolute direction of the directional indicator may change based on the change in orientation of the ultrasound device 102 relative to the operator processing device 104. However, in some embodiments, after a directional indicator is displayed, the processing device displaying the directional indicator may freeze the directional indicator's display in the user video 204 such that the position and orientation of the directional indicator do not change with changes in pose of the ultrasound device 102 relative to the operator processing device 104. In some embodiments, after a directional indicator is displayed, the processing device displaying the directional indicator may freeze the display of the directional indicator such that the orientation of the directional indicator does not change even as the orientation of the ultrasound device 102 relative to the operator processing device 104 changes, but the position of the directional indicator changes based on changes in position of the ultrasound device 102 relative to the operator processing device 104.

### Displaying Orientation Indicators

As described above, certain instruction interfaces may include orientation indicators (e.g., the orientation indicators 524 and 1354) that generally illustrate the direction the ultrasound device 102's marker 692 is pointing relative to the operator video 204. In particular, the position of the orientation indicator around a circle may change as the pose of the marker 692 on the ultrasound device 102 relative to the operator processing device 104 changes due to movement of the ultrasound device 102 relative to the operator processing device 104. FIG. 32 describes an example of how to display the orientation indicator in more detail.

FIG. 32 illustrates an example process 2900 for displaying an orientation indicator for an ultrasound device in an instruction interface, in accordance with certain embodiments described herein. The process 2900 may be performed by either the operator processing device 104 or the instructor processing device 122. For simplicity, the below description will describe the process 2900 as being performed by a processing device. All three-dimensional coordinates are given with the x-coordinate first, the y-coordinate second, and optionally the z-coordinate third (where x-, y-, and z-coordinates refer to position along the x-, y-, and z-axes, respectively, of the ultrasound device 102 in FIG. 27 relative to the origin 2509).

In act 2902, the processing device determines, based on a pose of the ultrasound device 102 to the operator processing device 104, two points in three-dimensional space along an axis of the ultrasound device 102. The pose of the ultrasound device 102 relative to the operator processing device 104 may have been determined using the methods described above. In some embodiments, the operator processing device 104 may determine a point P1 at (0, 0, 0), where point P1 is at a center of the ultrasound device 102, and a point P2 at (x, 0, 0), where x is any positive offset (e.g., 1) along the x-axis of the ultrasound device 102 and where, as illustrated in FIG. 27, the positive x-axis of the ultrasound device 102 is parallel to a line from the longitudinal axis of the ultrasound device 102 to the marker 692. The process 2900 proceeds from act 2902 to act 2904.

In act 2904, the processing device project the two points in three-dimensional space into two two-dimensional points in the operator video 204 captured by the operator processing device 104. In some embodiments, the processing device may rotate P2 by the three-dimensional rotation of the ultrasound device 102 relative to the operator processing device 104 (as determined using the methods described above for determining pose) with P1 being the origin of rotation. In some embodiments, the processing device may apply a rotation matrix to P2, where the rotation matrix describes the orientation of the ultrasound device 102 relative to the operator processing device 104. In some embodiments, the processing device may use camera intrinsics (e.g., focal lengths, skew coefficient, and principal points) to perform the projection. Let the coordinates of the projection of P1 be P1′ at (P1′ₓ, P1′_{y}) and the coordinates of the projection of P2 be P2′ at (P2′ₓ, P2′_{y}), where the first coordinate is along the horizontal axis of the operator video 204 and the second coordinate is along the vertical axis of the operator video 204. The process 2900 proceeds from act 2904 to act 2906.

In act 2906, the processing device display an orientation indicator at an angle relative to a horizontal axis of a display screen (although other axes may be used instead) that is equivalent to an angle between a line formed by the two two-dimensional points and a horizontal axis of the operator video 204 (although other axes may be used instead). In some embodiments, the processing device may determine a circle with center P1′ and with P2′ along the circumference of the circle. In other words, the distance between P1′ and P2′ is the radius of a circle. The processing device may determine a point P3 at (P1′ₓ+radius of the circle, P1′_{y}). In other words, P3 is on the circumference of the circle, directly offset to the right from P1′ in the operator video 204. The processing device may then calculate the angle between P1′-P3′ (i.e., a line extending between P1′ and P3′) and P1′-P2′ (i.e., a line extending between P1′ and P2′). This angle may be referred to as A. The processing device may display the orientation indicator around a circle in an instruction interface (e.g., the circle of the rotation interface 506, the tilt interface 806, or the translation interface 1006) such that the angle between a horizontal line through the circle (although other directions may be used instead) and a line extending between the center of the circle and the orientation indicator is A.

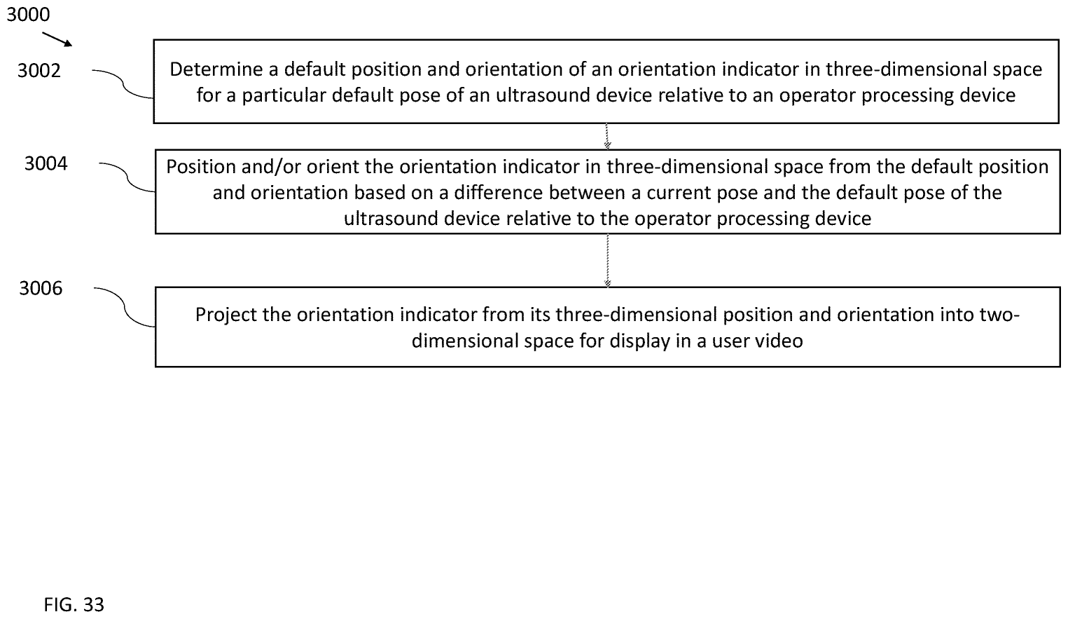

As described above, in some embodiments, the instructor GUI 300 may display an orientation indicator (e.g., the orientation ring 2607) including a ring (e.g., the ring 2603) and a ball (e.g., the ball 2605). The orientation ring 2607 may generally indicate in the operator video 204 the pose of the ultrasound device 102 relative to the operator processing device 104 and highlight the orientation of the marker 692 on the ultrasound device 102. The ring 2603 may be centered approximately at the tail of the ultrasound device 102 and oriented approximately within a plane orthogonal to the longitudinal axis of the ultrasound device 102. The ball 2605 may be located on the ring 2603 such that a line from the ring 2603 to the marker 692 on the ultrasound device 102 is parallel to the longitudinal axis of the ultrasound device 102. FIG. 33 describes an example of how to display this orientation indicator.

FIG. 33 illustrates an example process 3000 for displaying an orientation indicator for an ultrasound device in an operator video, in accordance with certain embodiments described herein. The process 3000 may be performed by either the operator processing device 104 or the instructor processing device 122. For simplicity, the below description will describe the process 3000 as being performed by a processing device.

In act 3002, the processing device determines a default position and orientation of the orientation indicator in three-dimensional space for a particular default pose of the ultrasound device 102 relative to the operator processing device 104. In this default position and orientation of the orientation indicator, the ring may be centered approximately at the tail of the ultrasound device 102 and oriented approximately within a plane orthogonal to the longitudinal axis of the ultrasound device 102, and the ball may be located on the ring such that a line from the ring to the marker 692 on the ultrasound device 102 is parallel to the longitudinal axis of the ultrasound device 102. The process 3000 proceeds from act 3002 to act 3004.

In act 3004, the processing device positions and/or orients the orientation indicator in three-dimensional space from the default position and orientation based on the difference between the current pose (as determined using the methods described above) and the default pose of the ultrasound device 102 relative to the operator processing device 104. The process 3000 proceeds from act 3004 to act 3006.

In act 3006, the processing device projects the orientation indicator from its three-dimensional position and orientation into two-dimensional space for display in the operator video 204. To perform this projection, the processing device may use camera intrinsics (e.g., focal lengths, skew coefficient, and principal points).

### Other Features

Referring back to FIG. 4, in response to a selection from the instructor of the draw option 416, the instructor GUI 300 may permit drawing on the ultrasound image 202 and/or the operator video 204. FIG. 34 illustrates an example of the instructor GUI 300, in accordance with certain embodiments described herein. The instructor GUI 300 in FIG. 34 is the same as the instructor GUI 300 in FIG. 3, except that the instructor GUI 300 in FIG. 34 includes a drawing 3196, an icon 3198, and a drawing 3199. The drawing 3196 and the icon 3198 are on the operator video 204 and the drawing 3199 is the one the ultrasound image 202. In some embodiments, in response to selection by the instructor (e.g., by touching a finger or a stylus to a screen or by clicking a mouse button) of a location of either the operator video 204 or the ultrasound image 202, the icon 3198 may appear. As the instructor continues to drag (e.g., by dragging a finger, stylus, or mouse while holding the mouse button), the icon 3198 may move corresponding to the dragging movement and trace a drawing. FIG. 34 illustrates the drawing 3196 created on the operator video 204 by dragging the icon 3198, and the drawing 3199 that was previously created on the ultrasound image. The instructor processing device 122 may output information regarding such drawings to the operator processing device 104 for display on the operator GUI 20.

FIG. 35 illustrates an example of the operator GUI 200, in accordance with certain embodiments described herein. The operator GUI 200 in FIG. 35 is the same as the operator GUI 200 in FIG. 2, except that the operator GUI 200 in FIG. 35 includes the drawing 3196 and the drawing 3198. The operator processing device 104 may display the drawing 3196 and the drawing 3198 in response to receiving information regarding these drawings from the instructor processing device 122. Such drawings may convey information from the instructor to the operator. For example, the drawing 3196 may instruct the operator to move the ultrasound device 102 to the location on the subject 208 highlighted by the drawing 3196 in the operator video 204. The drawing 3198 may highlight a feature of the ultrasound image 202 for the operator.

Referring back to FIG. 2, the operator GUI 200 further includes a freeze option 240, a record option 242, a preset option 244, a mode option 246, an operator indicator 232, an exam reel button 247, an information bar 248, a hang-up option 276, a mute option 277, and a further options button 275. In some embodiments, in response to receiving a selection of the freeze option 240, the operator processing device 104 may not update the ultrasound image 202 currently displayed on the operator GUI 200 and not transmit to the instructor processing device 122 new ultrasound images based on new ultrasound data collected by the ultrasound device 102. In some embodiments, in response to receiving a selection of the record option 242, the operator processing device 104 may save to memory ultrasound images as they are generated from ultrasound data collected by the ultrasound device 102. In some embodiments, in response to receiving a selection of the preset option 244, the operator processing device 104 may display a menu of presets (e.g., cardiac, abdominal, etc.). In some embodiments, in response to receiving a selection of a preset from the menu of presets, the operator processing device 104 may configure the ultrasound device 102 with imaging parameter values for the selected preset. In some embodiments, in response to receiving a selection of the mode option 246, the operator processing device 104 may display a menu of modes (e.g., B-mode, M-mode, color Doppler, etc.). In some embodiments, in response to receiving a selection of a mode from the menu of modes, the operator processing device 104 may configure the ultrasound device 102 to operate in the selected mode.

In some embodiments, the operator indicator 232 may include an indicator (e.g., initials or an image) of the operator of the ultrasound device 102. In some embodiments, in response to receiving a selection of the exam reel button 247, the operator GUI 200 may display an interface for interacting with ultrasound data captured during the session. The exam reel button 247 may show the number of sets of ultrasound data saved during the session. In some embodiments, the information bar 248 may display information related to the time, date, wireless network connectivity, and battery charging status. In some embodiments, in response to receiving a selection of the hang-up option 276, the operator processing device 104 may terminate its communication with the instructor processing device 122. In some embodiments, in response to receiving a selection of the mute option 277, the operator processing device 104 may not transmit audio to the instructor processing device 122. In some embodiments, in response to receiving a selection of the further options button 275, the operator GUI 200 may show further options (or display a new GUI with further options). In some embodiments, the instructor video 212 may depict the instructor. The instructor video 212 may be captured by a front-facing camera on the instructor processing device 122. The operator processing device 104 may receive the instructor video 212 from the instructor processing device 122. In some embodiments, rather than display the instructor video 212, the operator GUI 200 may display an instructor indicator (e.g., initials or an image).

Referring back to FIG. 3, the instructor GUI 300 further includes the instructor video 212, a freeze option 340, a record option 342, a preset option 344, a mode option 346, a gain and depth option 349, an instructor indicator 332, the exam reel button 247, the information bar 248, a hang-up option 376, a mute option 377, a video turn on-off option 336, a volume button 334, and a further options button 275.

In some embodiments, in response to receiving a selection of the freeze option 340, the instructor processing device 122 may issue a command to the operator processing device 104 to not update the ultrasound image 202 currently displayed on the operator GUI 200 and to not transmit to the instructor processing device 122 new ultrasound images based on new ultrasound data collected by the ultrasound device 102. In some embodiments, in response to receiving a selection of the record option 342, the instructor processing device 122 may issue a command to the operator processing device 104 to save to memory an ultrasound image or set of ultrasound images (e.g., cines) as they are generated from ultrasound data collected by the ultrasound device 102. In some embodiments, in response to receiving a selection of the preset option 344, the instructor processing device 122 may display a menu of presets (e.g., cardiac, abdominal, etc.). In some embodiments, in response to receiving a selection of a preset from the menu of presets, the instructor processing device 122 may issue a command to the operator processing device 104 to configure the ultrasound device 102 with imaging parameter values for the selected preset. In some embodiments, in response to receiving a selection of the mode option 346, the instructor processing device 122 may display a menu of modes (e.g., B-mode, M-mode, color Doppler, etc.). In some embodiments, in response to receiving a selection of a mode from the menu of modes, the instructor processing device 122 may issue a command to the operator processing device 104 to configure the ultrasound device 102 to operate in the selected mode. In some embodiments, in response to receiving a selection of the gain and depth option 349, the instructor processing device 122 may display an interface (e.g., a menu or a number pad) for inputting a gain or depth. In some embodiments, in response to receiving an input of a gain or depth, the instructor processing device 122 may issue a command to the operator processing device 104 to use this gain or depth for displaying subsequent ultrasound images 202 on the operator GUI 200. In some embodiments, the instructor processing device 122 may directly use the selected gain for displaying subsequent ultrasound images 202, while in other embodiments, subsequent ultrasound images 202 received from the operator processing device 104 may already use the selected gain. Thus, the instructor may control the ultrasound device 102 through the instructor GUI 300.

In some embodiments, the instructor indicator 332 may include an indicator (e.g., initials or image) of the instructor. In some embodiments, in response to receiving a selection of the mute option 377, the instructor processing device 122 may not transmit audio to the operator processing device 104. In some embodiments, in response to receiving a selection of the volume option 334, the instructor processing device 122 may modify the volume of audio output from its speakers. In some embodiments, in response to receiving a selection of the video turn-off option 336, the instructor processing device 122 may cease to transmit video from its camera to the operator processing device 104. In some embodiments, in response to receiving a selection of the hang-up option 376, the instructor processing device 122 may terminate its communication with the operator processing device 104. In some embodiments, in response to receiving a selection of the exam reel button 247, the instructor GUI 300 may display an interface for interacting with ultrasound data captured during the session.

According to an aspect of the present disclosure, a method is provided that comprises determining a pose of an ultrasound device relative to the operator processing device; receiving, from an instructor processing device, an instruction for moving the ultrasound device; and displaying, in an operator video displayed on the operator processing device, based on the pose of the ultrasound device relative to the operator processing device and based on the instruction, a directional indicator for moving the ultrasound device.

In one embodiment, the operator video depicts the ultrasound device.

In one embodiment, the directional indicator displayed in the operator video comprises an augmented reality display.

In one embodiment, the directional indicator is displayed in the operator video such that the directional indicator appears to be a part of a real-world environment in the operator video.

In one embodiment, the operator video is captured by a camera of the operator processing device.

In one embodiment, the instruction comprises an instruction to rotate, tilt, or translate the ultrasound device.

According to another aspect of the present disclosure, a method is provided that comprises receiving a pose of an ultrasound device relative to an operator processing device; and displaying, in an operator video displayed on an instructor processing device, based on the pose of the ultrasound device relative to the operator processing device, an orientation indicator indicating the pose of the ultrasound device relative to the operator processing device.

In one embodiment, the operator video depicts the ultrasound device.

In one embodiment, the directional indicator displayed in the operator video comprises an augmented reality display.

In one embodiment, the directional indicator is displayed in the operator video such that the directional indicator appears to be a part of a real-world environment in the operator video.

In one embodiment, the operator video is captured by a camera of the operator processing device.

In one embodiment, the orientation indicator indicates an orientation of a marker on the ultrasound device relative to the operator processing device.

According to another aspect of the present disclosure, a method is provided that comprises receiving a pose of an ultrasound device relative to an operator processing device; and displaying, in an instruction interface displayed on an instructor processing device, based on the pose of the ultrasound device relative to the operator processing device, an orientation indicator indicating the pose of the ultrasound device relative to the operator processing device.

In one embodiment, the orientation indicator illustrates a direction a marker on the ultrasound device is pointing relative to the operator processing device.

In one embodiment, the orientation indicator illustrates two-dimensionally a three-dimensional pose of a marker on the ultrasound device.

According to another aspect of the present disclosure, a method is provided that comprises receiving a pose of an ultrasound device relative to an operator processing device; receiving a selection of an instruction for moving the ultrasound device from an instruction interface; and displaying, in an operator video displayed on an instructor processing device, based on the pose of the ultrasound device relative to the operator processing device and based on the instruction, a directional indicator for moving the ultrasound device.

In one embodiment, the operator video depicts the ultrasound device.

In one embodiment, the directional indicator displayed in the operator video comprises an augmented reality display.

In one embodiment, the directional indicator is displayed in the operator video such that the directional indicator appears to be a part of a real-world environment in the operator video.

In one embodiment, the operator video is captured by a camera of the operator processing device.

In one embodiment, the instruction comprises an instruction to rotate, tilt, or translate the ultrasound device.

According to another aspect of the present disclosure, a method is provided that comprises displaying, on an instructor processing device, an instruction interface for selecting an instruction to translate an ultrasound device, the instruction interface comprising a rotatable arrow.

In one embodiment, the method further comprises receiving, from the instructor processing device, a selection of an instruction to translate the ultrasound device from the instruction interface based on an angle of the rotatable arrow.

In one embodiment, the instruction interface includes an orientation indicator indicating a pose of the ultrasound device relative to an operator processing device.

In one embodiment, the orientation indicator illustrates a direction a marker on the ultrasound device is pointing relative to the operator processing device.

In one embodiment, the orientation indicator illustrates two-dimensionally a three-dimensional pose of a marker on the ultrasound device.

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having described above several aspects of at least one embodiment, it is to be appreciated various alterations, modifications, and improvements will readily occur to those skilled in the art. Such alterations, modifications, and improvements are intended to be object of this disclosure. Accordingly, the foregoing description and drawings are by way of example only.

What is claimed is:

1. An ultrasound system, comprising:
an instructor processing device configured to:
receive a current pose of an ultrasound device relative to an operator processing device from the operator processing device; and
display, in an operator video displayed on the instructor processing device, based on the current pose of the ultrasound device relative to the operator processing device, an orientation indicator indicating the current pose of the ultrasound device relative to the operator processing device.

2. The ultrasound system of claim 1, wherein the operator video depicts the ultrasound device.

3. The ultrasound system of claim 1, wherein the orientation indicator is displayed in the operator video such that the orientation indicator appears to be a part of a real-world environment in the operator video.

4. The ultrasound system of claim 1, wherein the instructor processing device is configured, when displaying the orientation indicator indicating the current pose of the ultrasound device relative to the operator processing device, to:
determine a default position and orientation of the orientation indicator in three-dimensional space for a default pose of the ultrasound device relative to the operator processing device;
position and/or orient the orientation indicator in three-dimensional space from the default position and orientation based on a difference between the current pose and the default pose of the ultrasound device relative to the operator processing device; and
project the orientation indicator from its three-dimensional position and orientation into two-dimensional space for display in the operator video displayed on the instruction processing device.

5. The ultrasound system of claim 1, wherein the orientation indicator indicates an orientation of a marker on the ultrasound device relative to the operator processing device.

6. The ultrasound system of claim 5, wherein:
the orientation indicator comprises a ring and a ball;
the ring is centered in the operator video approximately at a tail of the ultrasound device and orientated approximately within a plane orthogonal to a longitudinal axis of the ultrasound device; and
the ball is located in the operator video on the ring such that a line from the ring to the marker on the ultrasound device is approximately parallel to the longitudinal axis of the ultrasound device.

7. An ultrasound system, comprising:
an instructor processing device configured to:
receive a current pose of an ultrasound device relative to an operator processing device from the operator processing device; and
display, in an instruction interface displayed on a display screen of the instructor processing device, based on the pose of the ultrasound device relative to the operator processing device, an orientation indicator indicating the current pose of the ultrasound device relative to the operator processing device.

8. The ultrasound system of claim 7, wherein the instructor processing device is configured, when displaying the orientation indicator indicating the current pose of the ultrasound device relative to the operator processing device, to:
determine, based on the current pose of the ultrasound device relative to the operator processing device, two points in three-dimensional space along an axis of the ultrasound device;
project the two points in three-dimensional space into two two-dimensional points in an operator video captured by the operator processing device; and
display the orientation indicator at an angle relative to a horizontal axis of the display screen of the instructor processing device that is equivalent to an angle between a line formed by the two two-dimensional points and the horizontal axis of the display screen of the instructor processing device.

9. The ultrasound system of claim 7, wherein the orientation indicator illustrates a direction a marker on the ultrasound device is pointing relative to the operator processing device.

10. The ultrasound system of claim 7, wherein the orientation indicator illustrates two-dimensionally a three-dimensional pose of a marker on the ultrasound device.

11. The ultrasound system of claim 7, wherein the instruction interface comprises a circle, and the orientation indicator is at a particular position around the circle that indicates the current pose of the ultrasound device relative to the operator processing device.

12. An ultrasound system, comprising:
an instructor processing device configured to:
display a translation instruction interface for selecting an instruction to translate an ultrasound device, the translation instruction interface comprising an arrow rotatable by an instructor.

13. The ultrasound system of claim 12, wherein the instructor processing device is further configured to:
receive a selection of an instruction to translate the ultrasound device from the translation instruction interface based on an angle of the arrow rotatable by the instructor.

14. The ultrasound system of claim 12, wherein the translation instruction interface includes an orientation indicator indicating a pose of the ultrasound device relative to an operator processing device.

15. The ultrasound system of claim 14, wherein the orientation indicator illustrates a direction a marker on the ultrasound device is pointing relative to the operator processing device.

16. The ultrasound system of claim 14, wherein the orientation indicator illustrates two-dimensionally a three-dimensional pose of a marker on the ultrasound device.

17. The ultrasound system of claim 12, wherein the instruction to translate the ultrasound device is based on an angle formed by the arrow rotatable by the instructor.

18. The ultrasound system of claim 12, further comprising:
an operator processing device configured to:
display, in an operator video captured by the operator processing device and displayed on an operator processing device, a two-dimensional arrow indicating a direction for translating the ultrasound device, wherein displaying the arrow comprises:
determine, based on a pose of the ultrasound device relative to the operator processing device, two points in three-dimensional space along an axis of the ultrasound device;
project the two points in three-dimensional space into two two-dimensional points in the operator video;
calculate an angle between a line formed by the two points and a horizontal axis of the operator video;
subtract the angle from a desired instruction angle to produce a final angle, wherein the desired instruction angle is determined from the arrow rotatable by the instructor on the translation instruction interface displayed on the instructor processing device;
determine, based on the pose of the ultrasound device relative to the operator processing device, an arrow in three-dimensional space pointing along the final angle; and
project the arrow in three-dimensional space into the two-dimensional arrow displayed in the operator video and indicating the direction for translating the ultrasound device.

19. The ultrasound system of claim 12, wherein the instructor processing device is further configured to display:
a rotation instruction interface for selecting an instruction to rotate the ultrasound device; and
a tilt instruction interface for selecting an instruction to tilt the ultrasound device.

20. The ultrasound system of claim 12, wherein the instructor processing device is further configured to receive a manually drawing from the instructor on an ultrasound image displayed by the instructor processing device.
